(19) United States
(12) Patent Application Publication
(10) Pub. No.: US 2008/0182872 A1
(43) Pub. Date: Jul. 31, 2008

(54) USE OF ER-BETA SELECTIVE LIGANDS FOR TREATING ACUTE LUNG INJURIES

(75) Inventors: James C. KEITH, Andover, MA (US); George P. VLASUK, Concord, MA (US)

Correspondence Address: Pepper Hamilton LLP/Wyeth, 400 Berwyn Park, 899 Cassatt Road, Berwyn, PA, 19312-1183, US

(73) Assignee: WYETH, Madison, NJ (US)

(21) Appl. No.: 12/022,348

(22) Filed: Jan. 30, 2008

Related U.S. Application Data
(60) Provisional application No. 60/887,400, filed on Jan. 31, 2007.

(51) Int. Cl. A61K 31/47; A61K 31/423; A61K 31/352; A61K 31/055

(52) U.S. Cl. 514/311; 514/375; 514/728; 514/453

(57) ABSTRACT

The present invention provides a method of treating acute lung injuries, such as acute lung injury induced by peritonitis during sepsis, acute lung injury induced by intravenous bacteremia during sepsis, acute lung injury caused by smoke inhalation, acute lung injury occurring in a premature infant with deficiency of surfactant, acute lung injury caused by oxygen toxicity or acute lung injury caused by barotraumas from mechanical ventilation, using an ERβ selective ligand such as 2-(3-fluoro-4-hydroxyphenyl)-7-vinyl-1,3-benzoxazol-5-ol or 3-(3-fluoro-4-hydroxy-phenyl)-7-hydroxy-naphthalene-1-carbonitrile. The present invention further relates to use of ERβ selective ligands or compositions thereof for the prevention of acute lung injuries in those being at risk thereof.

This application claims benefit of priority to U.S. provisional patent application Ser. No. 60/887,400 filed Jan. 31, 2007, which is hereby incorporated by reference in its entirety.

### FIELD OF THE INVENTION

The present invention relates, in part, to use of ERβ selective ligands or compositions thereof for the treatment of acute lung injuries, such as acute lung injury induced by peritonitis during sepsis, acute lung injury induced by intravenous bacteremia during sepsis, acute lung injury caused by smoke inhalation, acute lung injury occurring in a premature infant with deficiency of surfactant, acute lung injury caused by oxygen toxicity or acute lung injury caused by barotrauma from mechanical ventilation. In some embodiments, the ERβ selective ligand is administered orally or intravenously. In some embodiments, the ERβ selective ligand is non-uterotrophic, non-mammotrophic, or non-uterotrophic and non-mammotrophic. In some further embodiments, the ERβ selective ligand used is 2-(3-fluoro-4-hydroxyphenyl)-7-vinyl-1,3-benzoxazol-5-ol or 3-(3-fluoro-4-hydroxy-phenyl)-7-hydroxy-naphthalene-1-carbonitrile, or a pharmaceutically acceptable salt thereof. The present invention further relates to use of ERβ selective ligands or compositions thereof for the prevention of acute lung injuries.

### BACKGROUND OF THE INVENTION

The present invention relates to use of ERβ selective ligands for the treatment of acute lung injuries, such as acute lung injury induced by peritonitis during sepsis, acute lung injury induced by intravenous bacteremia during sepsis, acute lung injury caused by smoke inhalation, acute lung injury occurring in a premature infant with deficiency of surfactant, acute lung injury caused by oxygen toxicity or acute lung injury caused by barotrauma from mechanical ventilation.

The pleiotropic effects of estrogens in mammalian tissues have been well documented, and it is now appreciated that estrogens affect many organ systems [Mendelsohn and Karas, New England Journal of Medicine 340: 1801-1811 (1999), Epperson, et al., Psychosomatic Medicine 61: 676-697 (1999), Crandall, Journal of Womens Health & Gender Based Medicine 8: 1155-1166 (1999), Monk and Brodaty, Dementia & Geriatric Cognitive Disorders 11: 1-10 (2000), Hum and Macrae, Journal of Cerebral Blood Flow & Metabolism 20: 631-652 (2000), Calvin, Maturitas 34: 195-210 (2000), Finking, et al., Zeitschrift fur Kardiologie 89: 442-453 (2000), Brincat, Maturitas 35: 107-117 (2000), Al-Azzawi, Postgraduate Medical Journal 77: 292-304 (2001)]. Estrogens can exert effects on tissues in several ways, and the most well characterized mechanism of action is their interaction with estrogen receptors leading to alterations in gene transcription. Estrogen receptors (ER) are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences [Moggs and Orphanides, EMBO Reports 2: 775-781 (2001), Hall, et al., Journal of Biological Chemistry 276: 36869-36872 (2001), McDonnell, Principles Of Molecular Regulation. p 351-361(2000)]. A class of “coregulatory” proteins can also interact with the ligand-bound receptor and further modulate its transcriptional activity [McKenna, et al., Endocrine Reviews 20: 321-344 (1999)]. It has also been shown that estrogen receptors can suppress NFκB-mediated transcription in both a ligand-dependent and independent manner [Quaedackers, et al., Endocrinology 142: 1156-1166 (2001), Bhat, et al., Journal of Steroid Biochemistry & Molecular Biology 67: 233-240 (1998), Pelzer, et al., Biochemical & Biophysical Research Communications 286: 1153-7 (2001)].

Estrogen receptors can also be activated by phosphorylation. This phosphorylation is mediated by growth factors such as EGF and causes changes in gene transcription in the absence of ligand [Moggs and Orphanides, EMBO Reports 2: 775-781 (2001), Hall, et al., Journal of Biological Chemistry 276: 36869-36872 (2001)].

A less well-characterized means by which estrogens can affect cells is through a so-called membrane receptor. The existence of such a receptor is controversial, but it has been well documented that estrogens can elicit very rapid non-genomic responses from cells. The molecular entity responsible for transducing these effects has not been definitively isolated, but there is evidence to suggest it is at least related to the nuclear forms of the estrogen receptors [Levin, Journal of Applied Physiology 91: 1860-1867 (2001), Levin, Trends in Endocrinology & Metabolism 10: 374-377 (1999)].

Two estrogen receptors have been discovered to date. The first estrogen receptor was cloned about 15 years ago and is now referred to as ERα [Green, et al., Nature 320: 134-9 (1986)]. The second form of the estrogen receptor was found comparatively recently and is called ERβ [Kuiper, et al., Proceedings of the National Academy of Sciences of the United States of America 93: 5925-5930 (1996)]. Early work on ERβ focused on defining its affinity for a variety of ligands and indeed, some differences with ERα were seen. The tissue distribution of ERβ has been well mapped in the rodent and it is not coincident with ERα. Tissues such as the mouse and rat uterus express predominantly ERα, whereas the mouse and rat lung express predominantly ERβ [Couse, et al., Endocrinology 138: 4613-4621 (1997), Kuiper, et al., Endocrinology 138: 863-870 (1997)]. Even within the same organ, the distribution of ERα and ERβ can be compartmentalized. For example, in the mouse ovary, ERβ is highly expressed in the granulosa cells and ERα is restricted to the thecal and stromal cells [Sar and Welsch, Endocrinology 140: 963-971 (1999), Fitzpatrick, et al., Endocrinology 140: 2581-2591 (1999)]. However, there are examples where the receptors are coexpressed and there is evidence from in vitro studies that ERα and ERβ can form heterodimers [Cowley, et al., Journal of Biological Chemistry 272: 19858-19862 (1997)].

A large number of compounds have been described that either mimic or block the activity of 17β-estradiol. Compounds having roughly the same biological effects as 17β-estradiol, the most potent endogenous estrogen, are referred to as “estrogen receptor agonists”. Those which, when given in combination with 17β-estradiol, block its effects are called “estrogen receptor antagonists”. In reality there is a continuum between estrogen receptor agonist and estrogen receptor antagonist activity and indeed some compounds behave as estrogen receptor agonists in some tissues and estrogen receptor antagonists in others. These compounds with mixed activity are called selective estrogen receptor modulators (SERMS) and are therapeutically useful agents (e.g. EVISTA) [McDonnell, Journal of the Society for Gynecologic Investigation 7: S10-S15 (2000), Goldstein, et al., Human Reproduction Update 6: 212-224 (2000)]. The precise reason why the same compound can have cell-specific effects has not been elucidated, but the differences in receptor conformation and/or in the milieu of coregulatory proteins have been suggested.

It has been known for some time that estrogen receptors adopt different conformations when binding ligands. However, the consequence and subtlety of these changes has been only recently revealed. The three dimensional structures of ERα and ERβ have been solved by co-crystallization with various ligands and clearly show the repositioning of helix 12 in the presence of an estrogen receptor antagonist which sterically hinders the protein sequences required for receptor-coregulatory protein interaction [Pike, et al., Embo 18: 4608-4618 (1999), Shiau, et al., Cell 95: 927-937 (1998)]. In addition, the technique of phage display has been used to identify peptides that interact with estrogen receptors in the presence of different ligands [Paige, et al., Proceedings of the National Academy of Sciences of the United States of America 96: 3999-4004 (1999)]. For example, a peptide was identified that distinguished between ERα bound to the full estrogen receptor agonists 17β-estradiol and diethylstilbesterol. A different peptide was shown to distinguish between clomiphene bound to ERα and ERβ. These data indicate that each ligand potentially places the receptor in a unique and unpredictable conformation that is likely to have distinct biological activities.

Estrogens have been shown to have anti-inflammatory properties in a number of preclinical models [Vegeto E, et al, Proceedings of the National Academy of Sciences of the United States of America 2003;100(16):9614-9619; Harnish D C, et al, American Journal of Physiology Gastrointestinal & Liver Physiology 2004;286(1):G118-G125.]. Estrogens can inhibit NFκB activity, a transcription factor central to the inflammation cascade [Tzagarakis-Foster C, et al, Journal of Biological Chemistry 2002;277(47):44772-44777; Evans M J, et al, Circulation Research 2001 ;89(9):823-830], and which may play a role in mucositis.

Early studies on the tissue distribution of ERβ suggested it was a good drug target and there was much initial optimism about its clinical utility [Nilsson S, et al, Trends in Endocrinology & Metabolism 1998;9(10):387-395.]. Understanding the relative contributions of ERα and ERβ to estrogen physiology has recently been advanced by the in vivo profiling of ERα and ERβ selective agonists [Harris H A, et al, Endocrinology 2002;143(11):4172-4177; Harris H A, et al, Endocrinology 2003;144(10):4241-9.]. These studies clearly show that ERα mediates the effects of estrogens on the uterus, skeleton and vasomotor instability. ERβ selective agonists, however, are active in several preclinical models of inflammation and have a dramatic positive effect on the colonic epithelium. Additionally, it has recently been shown that ERβ is the predominant receptor form in the oral mucosa. [Valimaa H, et al, J Endocrinol. 2004;180(1):55-62]. Not only the lung expresses predominantly ERβ, ERβ is expressed in the human lung at similar levels in both males and females[Fasco M J, et al., “gender-dependent expression of alpha and beta estrogen receptors inhuman nontumor and tumor lung tissue, “Mol Cell Endocrinol. 2002,188:125-40.

As mentioned above, estrogens affect a panoply of biological processes. In some instances, where gender differences have been described (e.g. disease frequencies, responses to challenge, etc), it is possible that the explanation involves the difference in estrogen levels between males and females. In some instances, where a particular subtype of estrogen receptor such as ERβ is expressed in the same tissue or organ at similar levels in both males and females, modulation of such particular subtype of estrogen receptor will have the same effect in both males and females.

ERβ selective ligands are known to those of skilled in the art as compounds which preferentially bind to ERβ relative to ERα. The preparation of certain exemplary ERβ selective ligands, including 2-(3-fluoro-4-hydroxyphenyl)-7-vinyl-1,3-benzoxazol-5-ol (ERB-041), is described in U.S. Pat. No. 6,794,403, and WO 03/050095, each of which is incorporated herein by reference in its entirety. Further ERβ selective ligands [e.g., 3-(3-fluoro-4-hydroxy-phenyl)-7-hydroxy-naphthalene-1-carbonitrile] include compounds set forth in U.S. Pat. No. 6,794,403, U.S. Pat. No. 6,914,074; and U.S. Patent Application Ser. No 60/637,144, filed Dec. 17, 2004, each of which is incorporated herein by reference in its entirety. In addition, some pharmaceutical compositions containing ERβ selective ligands are described in U.S. Patent Application Ser. No 60/773,028, filed Feb. 14, 2006, incorporated herein by reference in its entirety.

Estrogen receptor beta (ERβ) is expressed in the lung at similar levels in both males and females. Upon binding to its ligand, ER-β mediates a number of cytosolic and transcriptional effects that may protect the host in pro-inflammatory conditions [Cristofaro, P. A., et al., “WAY-202196, a selective estrogen receptor-beta agonist, protects against death in experimental septic shock,” Crit Care Med 2006 Vol. 34, No. pages 2188-93; Hsieh, Y.-C., “upregulation of mitochondrial respiratory complex IV by estrogen receptor-β is critical for inhibiting mitochondrial apoptotic signaling and restoring cardiac functions following trauma-hemorrhage,” Journal of Molecular and Cellular Cardiology, 41 (2006), 511-521.]. Some examples of pro-inflammatory conditions include acute lung injuries, such as acute lung injury induced by peritonitis during sepsis, acute lung injury induced by intravenous bacteremia during sepsis, acute lung injury caused by smoke inhalation, acute lung injury occurring in a premature infant with deficiency of surfactant, acute lung injury caused by oxygen toxicity or acute lung injury caused by barotrauma from mechanical ventilation.

Systemic infection and pneumococcal sepsis following airway infection with S. pneumoniae remains a major cause of morbidity and mortality in the United States. Streptococcus pneumoniae remains the most common cause of severe community-acquired pneumonia in the United States, despite decades of effective antimicrobial therapy and pneumococcal vaccination. The mortality rate remains in the 15-25 percent range. [The National Heart, Lung and Blood Institute Acute Respiratory Distress Syndrome Clinical Trials Network. N Engl J Med 2006; 354:2564-2575.] A pronounced and persistent local and systemic inflammatory response exists in bacterial pneumonia complicated by prothrombotic events, diminished fibrinolytic activity, tissue damage and loss of organ function. Adjuvant strategies have been sought for many years to assist in the management of pneumococcal pneumonia.

The pathogenesis of sepsis involves a complex process of cellular activation resulting in the release of proinflammatory mediators, such as cytokines, activation of neutrophils, monocytes, and microvascular endothelial cells, involvement of neuroendocrine reflexes, and activation of the complement, coagulation, and fibrinolytic systems. [“The Last 100 Years of Sepsis,” Vincent, J-L, et al., American Journal of Respiratory and Critical Care Medicine Vol.173, pages 256-263, 2006]

The pulmonary and vascular injury in acute lung injury (ALI) associated with sepsis is primarily mediated by activated leukocytes (Murakami K, Okajima K, Uchiba M, et al., “Activated protein C attenuates endotoxininduced pulmonary vascular injury by inhibiting activated leukocytes in rats”; Blood; 1996; 87:642-647) as well as fibrin formation in the airway and pulmonary microvasculature (Gunther A, Mosavi P, Heinemann S, et al.; “Alveolar fibrin formation caused by enhanced procoagulant and depressed fibrinolytic capacities in severe pneumonia: Comparison with the acute respiratory distress syndrome”; Am J Respir Crit Care Med 2000; 161:454-462). [Maybauer, M. O., et al., “recombinant human activated protein C improves pulmonary function in ovine acute lung injury resulting from smoke inhalation and sepsis”; Crit. Care Med., 2006, Vol. 34, No. 9, pages 2432-38]

Severe smoke inhalation commonly results in acute respiratory distress syndrome (ARDS), which is frequently associated with superinfection, resulting in further exacerbation of the severity of acute lung injury. Pseudomonas aeruginosa pneumonia is frequently observed after smoke inhalation, and pneumonia is a frequent cause of sepsis in these patients [Maybauer, M. O., et al., “recombinant human activated protein C improves pulmonary function in ovine acute lung injury resulting from smoke inhalation and sepsis”; Crit. Care Med., 2006, Vol. 34, No. 9, pages 2432-38].

The premature infants are disadvantaged from a respiratory viewpoint from the time of delivery. Lung immaturity, coupled with impaired surfactant production, results in widespread atelectasis and ventilation/perfusion inequality. The ability to meet this increase in respiratory work is potentially compromised by an immature central drive and a highly compliant chest wall. Increasing levels of supplemental oxygen and assisted ventilation are needed to maintain adequate oxygenation. This is a catalyst for a host response of increasing inflammatory change with platelet, neutrophil and pulmonary alveolar macrophage activation. Pro-inflammatory cytokines (TNF, 1 L-1, 1 L6), eiconsanoids, chemokines (1 L8, macrophage inflammatory protein) are released in addition to oxygen free radicals, elastase and fibronectin. Immaturity of the intracellular antioxidant system and imbalance of elastase-α1-proteinase inhibitor system results in further lung injury. There then follows a reparative process with suppression of the inflammatory cascade through feedback loops and alterations in the levels of anti-inflammatory mediators. [Kennedy, J. D.; “lung function outcome in children of premature birth”; J. Paediatr. Child Health (1999) 35, 516-521.] The major long-term respiratory complication of a preterm birth is bronchopulmonary dysplasia (BPD), originally described by Northway et al. [Northway W H Jr, et al., “pulmonary disease following respirator therapy of hyaline-membrane disease. Bronchopulmonary dysplasia”; N. Engl. J. Med. 1967; 276: 357-68.] The term chronic lung disease (CLD) was introduced by Tooley and has now come to imply the need for supplemental oxygen at a corrected age of 36 weeks. [Tooley, W. H., “epidemiology of bronchopulmonary dysplasia,” J. Pediatr. 1979, 95: 851-8; and Shennan A. T., et al., “abnormal pulmonary outcomes in premature infants: Prediction from oxygen requirement in the neonatal period,” Pediatrics, 1988, 82: 527-32.]

Because new and improved methods for treating medical conditions such as acute lung jury are constantly sought, the present invention meet this and other important ends.

### SUMMARY OF THE INVENTION

In some embodiments, the present invention provides methods of treating acute lung injury in a subject in need thereof, comprising administering to the subject a therapeutically effective amount of an ERβ selective ligand or a pharmaceutical composition thereof. In some embodiments, the acute lung injury is acute lung injury induced by peritonitis during sepsis, acute lung injury induced by intravenous bacteremia during sepsis, acute lung injury caused by smoke inhalation, acute lung injury occurring in a premature infant with deficiency of surfactant, acute lung injury caused by oxygen toxicity or acute lung injury caused by barotrauma from mechanical ventilation.

In some embodiments, the acute lung injury is acute lung injury induced by peritonitis during sepsis, or acute lung injury induced by intravenous bacteremia during sepsis. In some embodiments, the acute lung injury is acute lung injury caused by smoke inhalation. In some embodiments, the acute lung injury is acute lung injury occurring in a premature infant with deficiency of surfactant. In some embodiments, the acute lung injury is acute lung injury caused by oxygen toxicity or acute lung injury caused by barotrauma from mechanical ventilation. In some embodiments, the acute lung injury is acute lung injury caused by oxygen toxicity occurring in a premature infant with deficiency of surfactant, or acute lung injury caused by barotrauma from mechanical ventilation occurring in a premature infant with deficiency of surfactant.

In some embodiments, the present invention provides methods of treating at least one symptom of acute lung injury in a subject in need thereof, comprising administering to said subject a therapeutically effective amount of an ERβ selective ligand or a pharmaceutical composition thereof. In some embodiments, the at least one symptom is selected from lung hemorrhage, hyaline membrane formation, and lung lesion. In some embodiments, the at least one symptom is selected from lung edema and lung inflammation.

In some embodiments, the present invention provides methods of preventing acute lung injury or at least one symptom of acute lung injury in a subject comprising administering to said subject a therapeutically effective amount of an ERβ selective ligand or a pharmaceutical composition thereof wherein said subject is suspected of being at risk for acute lung injury. In some embodiments, the present invention provides methods of preventing acute lung injury in a subject who is suspected of being at risk for acute lung injury. In some embodiments, the present invention provides methods of preventing at least one symptom of acute lung injury in a subject who is suspected of being at risk for acute lung injury. In some embodiments, the methods of preventing acute lung injury or at least one symptom of acute lung injury in the subject comprise identifying the subject who is suspected of being at risk for acute lung injury. In some further embodiments, identifying the subject who is suspected of being at risk for acute lung injury comprise diagnosing the subject. In some embodiments, the subject suspected of being at risk for acute lung injury is selected from a subject being suspected of being at risk for sepsis, a subject being suspected of being at risk for severe sepsis, a subject being suspected of being at risk for septic shock, a premature infant with deficiency of surfactant, a subject being suspected of being at risk for inhalation of noxious fumes, a subject being suspected of being at risk for burn, a subject being suspected of being at risk for massive blood transfusion, a subject being suspected of being at risk for acute pancreatitis, and a subject being suspected of being at risk for drug overdose. In some embodiments, the at least one symptom is selected from lung hemorrhage, hyaline membrane formation, pulmonary infiltrates, lung edema, lung inflammation, increased perivascular fluid flux, increased transvascular fluid flux, prevalent interstitial edema, alveolar collapse and increased respiratory rate.

In some embodiments, the ERβ selective ligand or the pharmaceutical composition thereof is administered orally. In some embodiments, the ERβ selective ligand or the pharmaceutical composition thereof is administered intravenously.

In some of each of the foregoing embodiments, the ERβ selective ligand is non-uterotrophic, non-mammotrophic, or non-uterotrophic and non-mammotrophic. In some embodiments of the foregoing methods, the subject is a human.

### DETAILED DESCRIPTION OF THE INVENTION

In some embodiments, the present invention provides methods of treating acute lung injury in a subject in need thereof, comprising administering to the subject a therapeutically effective amount of an ERβ selective ligand or a pharmaceutical composition thereof. “Acute lung injury” refers to a critical illness syndrome consisting of acute hypoxemic respiratory failure with bilateral pulmonary infiltrates that are not attributed to left atrial hypertension. [See e.g., Rubenfeld G D et al., “incidence and outcomes of acute lung injury,” N. Engl. J. Med. 2005, 353:1685-93.] “Acute lung injury” also refers to a syndrome of life-threatening progressive pulmonary insufficiency or hypoxemic respiratory failure in the absence of known pulmonary disease (such as emphysema, bronchitis, or chronic obstructive pulmonary disease), usually following a systemic insult such as surgery or major trauma. In some embodiments of the methods of the present invention, the acute lung injury is induced by diseases or disorders other than pulmonary diseases. In some embodiments of the methods of the present invention, the acute lung injury is pulmonary injury caused/induced by an extrapulmonary origin such as neurogenic pulmonary injury, secondary to severe CNS (central nervous system) trauma. In some embodiments of the methods of the present invention, the acute lung injury is acute lung injury induced by extrapulmonary diseases. In some embodiments of the methods of the present invention, the acute lung injury is indirect pulmonary injury from trauma, sepsis, and other disorders such as acute pancreatitis, drug overdose. In some embodiments of the methods of the present invention, the acute lung injury is acute lung injury induced by inhalation of noxious fumes, burn, or massive blood transfusion. In some embodiments of the methods of the present invention, the acute lung injury is acute lung injury induced by peritonitis during sepsis, acute lung injury induced by intravenous bacteremia during sepsis, acute lung injury caused by smoke inhalation, acute lung injury occurring in a premature infant with deficiency of surfactant, acute lung injury caused by oxygen toxicity or acute lung injury caused by barotrauma from mechanical ventilation.

In some embodiments, the acute lung injury is acute lung injury induced by peritonitis during sepsis, or acute lung injury induced by intravenous bacteremia during sepsis. In some embodiments, the acute lung injury is acute lung injury caused by smoke inhalation. In some embodiments, the acute lung injury is acute lung injury occurring in a premature infant with deficiency of surfactant. In some embodiments, the acute lung injury is acute lung injury caused by oxygen toxicity or acute lung injury caused by barotrauma from mechanical ventilation. In some embodiments, the acute lung injury is acute lung injury caused by oxygen toxicity occurring in a premature infant with deficiency of surfactant, or acute lung injury caused by barotrauma from mechanical ventilation occurring in a premature infant with deficiency of surfactant.

In some embodiments, the present invention provides methods of treating at least one symptom of acute lung injury in a subject in need thereof, comprising administering to said subject a therapeutically effective amount of an ERβ selective ligand or a pharmaceutical composition thereof. In some embodiments, the at least one symptom is selected from lung hemorrhage, hyaline membrane formation, and lung lesion. In some embodiments, the at least one symptom is selected from lung edema and lung inflammation. In some embodiments, the at least one symptom is selected from increased transvascular fluid flux, prevalent interstitial edema and alveolar collapse. In some embodiments, the at least one symptom is selected from prevalent interstitial edema and alveolar collapse.

In some embodiments, the present invention provides methods of preventing acute lung injury or at least one symptom of acute lung injury in a subject comprising administering to said subject a therapeutically effective amount of an ERβ selective ligand or a pharmaceutical composition thereof wherein said subject is suspected of being at risk for acute lung injury. In some embodiments, the present invention provides methods of preventing acute lung injury in a subject who is suspected of being at risk for acute lung injury. In some embodiments, the subject suspected of being at risk for acute lung injury is selected from a subject being suspected of being at risk for sepsis, a subject being suspected of being at risk for severe sepsis, a subject being suspected of being at risk for septic shock, a premature infant with deficiency of surfactant, a subject being suspected of being at risk for inhalation of noxious fumes, a subject being suspected of being at risk for burn, a subject being suspected of being at risk for massive blood transfusion, a subject being suspected of being at risk for acute pancreatitis, and a subject being suspected of being at risk for drug overdose. In some embodiments, the subject suspected of being at risk for acute lung injury is selected from a subject being suspected of being at risk for sepsis, a subject being suspected of being at risk for severe sepsis, a subject being suspected of being at risk for septic shock, and a premature infant with deficiency of surfactant. In some embodiments, the subject suspected of being at risk for acute lung injury is selected from a subject who has been previously diagnosed of sepsis, severe sepsis, or septic shock. In some embodiments, the subject suspected of being at risk for acute lung injury is a premature infant. In some embodiments, the subject suspected of being at risk for acute lung injury is a premature infant who is subject to supplemental oxygen, assisted ventilation, or supplemental oxygen and assisted ventilation. In some embodiments, the subject suspected of being at risk for acute lung injury is a premature infant with deficiency of surfactant. In some embodiments, the subject suspected of being at risk for acute lung injury is a premature infant with deficiency of surfactant who is subject to supplemental oxygen, assisted ventilation, or supplemental oxygen and assisted ventilation. In some embodiments, the subject suspected of being at risk for acute lung injury is selected from a subject being suspected of being at risk for inhalation of noxious fumes, burn, massive blood transfusion, acute pancreatitis, or drug overdose. In some embodiments, the subject suspected of being at risk for acute lung injury is selected from a subject being suspected of being at risk for inhalation of noxious fumes such as smoke in a fire. In some embodiments, the present invention provides methods of preventing at least one symptom of acute lung injury in a subject who is suspected of being at risk for acute lung injury. In some embodiments, the at least one symptom is selected from lung hemorrhage, hyaline membrane formation, pulmonary infiltrates, lung edema, lung inflammation, increased perivascular fluid flux, increased transvascular fluid flux, prevalent interstitial edema, alveolar collapse and increased respiratory rate.

In some of each of the foregoing embodiments, the ERβ selective ligand or the pharmaceutical composition thereof is administered orally. In some of each of the foregoing embodiments, the ERβ selective ligand or the pharmaceutical composition thereof is administered intravenously. In some of each of the foregoing embodiments, the ERβ selective ligand or the pharmaceutical composition thereof is administered via intravenous injection.

In some of each of the foregoing embodiments, the ERβ selective ligand is non-uterotrophic, non-mammotrophic, or non-uterotrophic and non-mammotrophic. In some of each of the foregoing embodiments, the ERβ selective ligand is an ERβ agonist (i.e., an ERβ selective agonist). In some embodiments of the foregoing methods, the subject is a human.

In some embodiments of the foregoing methods, the binding affinity of the ERβ selective ligand to ERβ is at least about 20 times greater than its binding affinity to ERα. In further embodiments, the binding affinity of the ERβ selective ligand to ERβ is at least about 50 times greater than its binding affinity to ERα.

In some further embodiments of the foregoing methods, the ERβ selective ligand causes an increase in wet uterine weight is less than about 25% of that observed for a maximally efficacious dose of 17β-estradiol in a standard pharmacological test procedure measuring uterotrophic activity, for example the uterotrophic test procedure as described herein.

In some further embodiments of the foregoing methods, the ERβ selective ligand causes an increase in defensin β1 mRNA which is less than about 25% of that observed for a maximally efficacious dose of 17β-estradiol in a standard pharmacological test procedure measuring mammotrophic activity, for example, the Mammary End Bud Test Procedure as described herein.

In some further embodiments of the foregoing methods, the ERβ selective ligand causes an increase in wet uterine weight which is less than about 10% of that observed for a maximally efficacious dose of 17β-estradiol in a standard pharmacological test procedure measuring uterotrophic activity. In some further embodiments, the ERβ selective ligand causes an increase in defensin β1 mRNA which is less than about 10% of that observed for a maximally efficacious dose of 17β-estradiol in a standard pharmacological test procedure measuring mammotrophic activity. In some embodiments, defensin β1 mRNA is detected using one or more of SEQ ID NO:1, SEQ ID NO:2 or SEQ ID NO:3.

In some further embodiments of the foregoing methods, the ERβ selective ligand does not significantly (p>0.05) increase wet uterine weight compared with a control that is devoid of uterotrophic activity, and does not significantly (p>0.05) increase defensin β1 mRNA compared with a control that is devoid of mammotrophic activity.

In some embodiments of the foregoing methods, the ERβ selective ligand has Formula I:

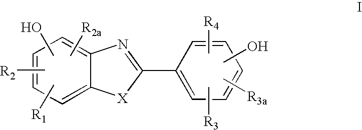

or is a pharmaceutically acceptable salt thereof, wherein:

R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms each independently selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₅, R₆ are each, independently, hydrogen, alkyl of 1-6 carbon atoms, aryl of 6-10 carbon atoms;

X is O, S, or NR₇;

R₇ is hydrogen, alkyl of 1-6 carbon atoms, aryl of 6-10 carbon atoms, —COR₅, —CO₂R₅ or —SO₂R₅.

In some embodiments of the foregoing methods, the ERβ selective ligand has Formula II:

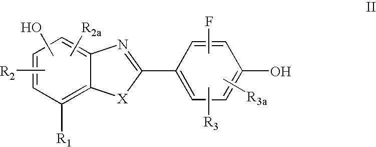

or is a pharmaceutically acceptable salt thereof, wherein:

R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;

R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR, —CO₂R₅, —NO₂, CONR₅R₆, NR₅R₆ or N(R₅)COR₆;

R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR, —CO₂R₅, —NO₂, CONR₅R₆, NR₅R₆ or N(R₅)COR₆;

R₅, R₆ are each, independently, hydrogen, alkyl of 1-6 carbon atoms, aryl of 6-10 carbon atoms;

X is O, S, or NR₇;

R₇ is hydrogen, alkyl of 1-6 carbon atoms, aryl of 6-10 carbon atoms, —COR₅, —CO₂R₅ or —SO₂R₅.

In some embodiments wherein the ERβ selective ligand has Formula II or is a pharmaceutically acceptable salt thereof, X is O.

In some embodiments wherein the ERβ selective ligand has Formula II or is a pharmaceutically acceptable salt thereof, R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆. In some further embodiments, R₁ is alkenyl of 2 carbon atoms (i.e., vinyl), which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆. In still further embodiments, R₁ is vinyl optionally substituted with —CN or halogen. In yet further embodiments, R₁ is vinyl.

In some embodiments wherein the ERβ selective ligand has Formula II or is a pharmaceutically acceptable salt thereof, X is O and R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆. In some further embodiments, R₁ is alkenyl of 2 carbon atoms (i.e., vinyl), which is optionally substituted with hydroxyl, —CN, halogen, trifluoroalkyl, trifluoroalkoxy, —COR, —CO₂R₅, —NO₂, CONR₅R₆, NR₅R₆ or N(R₅)COR₆. In still further embodiments, R₁ is vinyl optionally substituted with —CN or halogen. In yet further embodiments, R₁ is vinyl.

In some embodiments wherein the ERβ selective ligand has Formula II or is a pharmaceutically acceptable salt thereof, R₂ and R₂ₐ are each, independently, hydrogen, alkyl of 1-6 carbon atoms, or halogen. In some further embodiments, R₂ and R₂ₐ are each hydrogen. In yet further embodiments X is O and R₂ and R₂ₐ are each, independently, hydrogen, alkyl of 1-6 carbon atoms, or halogen. In still further embodiments, X is O and R₂and R₂ₐ are each hydrogen.

In some embodiments wherein the ERβ selective ligand has Formula II or is a pharmaceutically acceptable salt thereof, R₃ and R₃ₐ are each, independently, hydrogen or halogen. In some further embodiments, R₃and R₃ₐ are each hydrogen. In yet embodiments X is O and R₃ and R₃ₐ are each, independently, hydrogen or halogen. In still further embodiments, X is O and R₃ and R₃ₐ are each hydrogen. In some preferred embodiments of the foregoing methods, the ERβ selective ligand is a compound having the formula:

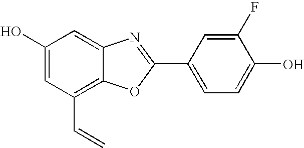

or a pharmaceutically acceptable salt thereof (Compound 2 or pharmaceutically acceptable salt thereof).

The preparation of compounds of Formulas I and II, including 2-(3-fluoro-4-hydroxyphenyl)-7-vinyl-1,3-benzoxazol-5-ol (ERB-041, Compound 2), is described in US Published Application 2003/0199562 (U.S. patent application Ser. No. 10/309,699 filed on Dec. 4, 2002), U.S. Pat. No. 6,794,403, and PCT/US2002/038513, filed Dec. 2, 2002, each of which is incorporated by reference herein in its entirety.

In some embodiments of the foregoing methods, the ERβ selective ligand has the Formula III:

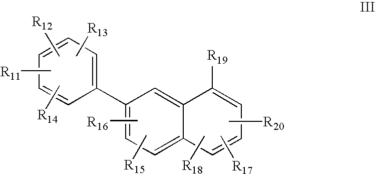

or a pharmaceutically acceptable salt thereof, wherein:

R₁₁, R₁₂, R₁₃, and R₁₄ are each, independently, selected from hydrogen, hydroxyl, alkyl of 1-6 carbon atoms, alkoxy of 1-6 carbon atoms, or halogen;

R₁₅, R₁₆, R₁₇, R₁₈, R₁₉, and R₂₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms each independently selected from O, N or S; wherein the alkyl or alkenyl moieties of R₁₅, R₁₆, R₁₇, R₁₈, R₁₉, or R₂₀ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈, R₁₉, or R₂₀ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;

wherein at least one of R₁₁, R₁₂, R₁₃, R₁₄, R₁₇, R₁₈, R₁₉ or R₂₀ is hydroxyl.

In some such embodiments, the ERβ selective ligand has the Formula IV:

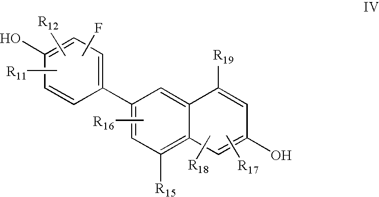

or a pharmaceutically acceptable salt thereof, wherein:

R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;

R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms each independently selected from O, N or S; wherein the alkyl or alkenyl moieties of R₁₅, R₁₆, R₁₇, R₁₈, or R₁₉ may be optionally substituted with hydroxyl, —CN, halogen, trifluoroalkyl, trifluoroalkoxy, —NO₂, or phenyl; wherein the phenyl moiety of R₁₅, R₁₆, R₁₇, R₁₈, or R₁₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;

wherein at least one of R₁₅ or R₁₉ is not hydrogen.

In some such embodiments, the ERβ selective ligand has the Formula V:

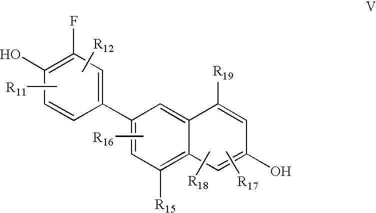

or is a pharmaceutically acceptable salt thereof, wherein:

R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;

R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms each independently selected from O, N or S; wherein the alkyl or alkenyl moieties of R₁₅, R₁₆, R₁₇, R₁₈, or R₁₉ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈ or R₉ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;

wherein at least one of R₁₅ or R₁₉ is not hydrogen.

In some embodiments of the foregoing methods wherein the ERβ selective ligand has Formula V, R₁₁ and R₁₂ are each, independently, selected from hydrogen and halogen. In some further embodiments, R₁₁ and R₁₂ are each hydrogen. In some embodiments of the foregoing methods wherein the ERβ selective ligand has Formula V, R₁₅ and R₁₉ are each, independently, hydrogen, alkyl of 1-6 carbon atoms, alkenyl of 2-7 carbon atoms, alkynyl of 2-7 carbon atoms, halogen, —CN, —CHO, trifluoromethyl; wherein each of the alkyl or alkenyl moieties of R₁₅ or R₁₉ may be optionally substituted with hydroxyl, —CN, halogen, trifluoroalkyl, trifluoroalkoxy, —NO₂, or phenyl. In some further embodiments, R₁₅ is alkyl of 1-6 carbon atoms, alkenyl of 2-7 carbon atoms, alkynyl of 2-7 carbon atoms, halogen, —CN, —CHO, or trifluoromethyl, wherein each of the alkyl or alkenyl moieties may be optionally substituted with hydroxyl, —CN, halogen, trifluoroalkyl, trifluoroalkoxy, —NO₂, or phenyl. In yet further embodiments, R₁₅ is —CN.

In some embodiments of the foregoing methods wherein the ERβ selective ligand has Formula V, R₁₉ is hydrogen, alkyl of 1-6 carbon atoms, alkenyl of 2-7 carbon atoms, alkynyl of 2-7 carbon atoms, halogen, —CN, —CHO, or trifluoromethyl, wherein each of the alkyl or alkenyl moieties may be optionally substituted with hydroxyl, —CN, halogen, trifluoroalkyl, trifluoroalkoxy, —NO₂, or phenyl. In some further embodiments, R₁₉ is hydrogen, —CN, or halogen. In yet further embodiments, R₁₅ is —CN, and R₁₉ is hydrogen or halogen.

In some embodiments of the foregoing methods wherein the ERβ selective ligand has Formula V, R₁₆, R₁₇, and R₁₈ are each, independently, hydrogen, alkyl of 1-6 carbon atoms, or halogen. In some further embodiments, R₁₆, R₁₇, and R₁₈ are each, independently, hydrogen or halogen.

In some embodiments wherein the ERβ selective ligand has Formula V, the 5 or 6-membered heterocyclic ring having 1 to 4 heteroatoms each independently selected from O, N or S is furan, thiophene or pyridine, and R₁₅, R₁₆, R₁₇, R₁₈, and R₁₉ are each, independently, hydrogen, halogen, —CN, or alkynyl of 2-7 carbon atoms. In some such embodiments, R₁₆, R₁₇, and R₁₈ are hydrogen. In some further embodiments of the foregoing methods, the ERβ selective ligand is the compound 3-(3-fluoro-4-hydroxy-phenyl)-7-hydroxy-naphthalene-1-carbonitrile (Compound 1), which has the formula:

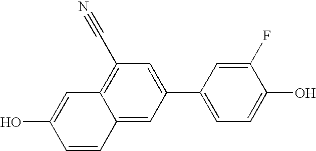

or a pharmaceutically acceptable salt thereof.

The preparation of compounds of Formulas III, IV and V is described in US Published Application 2003/0181519, U.S. Pat. No. 6,914,074, and PCT US 02/39883, filed Dec. 2, 2002, each of which is incorporated by reference herein in its entirety.

In some further embodiments of the foregoing methods, the ERβ selective ligand has the Formula VII:

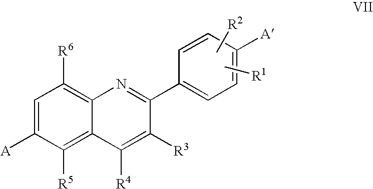

or a pharmaceutically acceptable salt thereof or a N-oxide thereof, wherein:
A and A′ are each, independently, OH or OP;
P is alkyl, alkenyl, benzyl, acyl, aroyl, alkoxycarbonyl, sulfonyl or phosphoryl;
R¹ and R² are each, independently, H, halogen, C₁-C₆ alkyl, C₂-C₇ alkenyl, or C₁-C₆ alkoxy;
R³ is H, halogen, or C₁-C₆ alkyl;
R⁴ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, or heteroaryl;
R⁵ and R⁶ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R⁴, R⁵ and R⁶ is halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl;
wherein the alkyl or alkenyl moieties of R⁴, R⁵ or R⁶ may be optionally substituted with halogen, OH, —CN, trifluoroalkyl, trifluoroalkoxy, —NO₂, or phenyl;
wherein the alkynyl moiety of R⁴, R⁵ or R⁶ may be optionally substituted with halogen, —CN, —CHO, acyl, trifluoroalkyl, trialkylsilyl, or optionally substituted phenyl;
wherein the phenyl moiety of R⁵ or R⁶ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R⁴, R⁵ and R⁶ are H, C₁-C₆ alkyl, C₂-C₇ alkenyl, or C₁-C₆ alkoxy, then at least one of R¹ and R² is halogen, C₁-C₆ alkyl, C₂-C₇ alkenyl, or C₁-C₆ alkoxy;
provided that at least one of R⁴ and R⁶ is other than H.

In some embodiments of the foregoing methods wherein the ERβ selective ligand has Formula VII, at least one of A and A′ is OH. In some further embodiments, A and A′ are each OH.

In some embodiments of the foregoing methods wherein the ERβ selective ligand has Formula VII, R¹, R², and R³ are each, independently H or halogen. In some further embodiments, at least one of R¹ and R² is halogen.

In some embodiments of the foregoing methods wherein the ERβ selective ligand has Formula VII, R⁴is H, halogen, C₁-C₆ alkyl, C₂-C₇ alkenyl, C₂-C₇ alkynyl, —CN, —CHO, or acyl. In some further embodiments, R⁴ is halogen, C₁-C₆ alkyl, C₂-C₇ alkenyl, C₂-C₇ alkynyl, or —CN. In yet further embodiments, R⁴ is halogen, C₂-C₇ alkynyl, or —CN.

In some embodiments of the foregoing methods wherein the ERβ selective ligand has Formula VII, R⁵is H, halogen, or CN. In some further embodiments, R⁵ is H.

In some embodiments of the foregoing methods wherein the ERβ selective ligand has Formula VII, R⁶ is H, halogen, C₁-C₆ alkyl, C₂-C₇ alkenyl, C₂-C₇ alkynyl, —CN, —CHO, acyl, or optionally substituted phenyl. In some further embodiments, R⁶ is H, halogen, C₁-C₆ alkyl, C₂-C₇ alkenyl, C₂-C₇ alkynyl, —CN, or optionally substituted phenyl. In yet further embodiments, R⁶ is halogen, C₂-C₇ alkynyl, or —CN.

In some embodiments of the foregoing methods wherein the ERβ selective ligand has Formula VII, R⁴ and R⁶ are each, independently, halogen, C₂-C₇ alkynyl, or —CN.

In some embodiments of the foregoing methods wherein the ERβ selective ligand has Formula VII, A and A′ are each OH; and R¹, R², and R³ are each, independently H or halogen.

In some embodiments of the foregoing methods wherein the ERβ selective ligand has Formula VII, A and A′ are each OH; and R⁵ is H, halogen, or CN.

In some embodiments of the foregoing methods wherein the ERβ selective ligand has Formula VII, A and A′ are each OH; R⁴ is H, halogen, C₁-C₆ alkyl, C₂-C₇ alkenyl, C₂-C₇ alkynyl, —CN, —CHO, or acyl; R⁶ is H, halogen, C₁-C₆ alkyl, C₂-C₇ alkenyl, C₂-C₇ alkynyl, —CN, —CHO, acyl, or optionally substituted phenyl; and R⁵ is H, halogen, or CN.

In some embodiments of the foregoing methods wherein the ERβ selective ligand has Formula VII, A and A′ are each OH; R⁴ is halogen, C₁-C₆ alkyl, C₂-C₇ alkenyl, C₂-C₇ alkynyl, or —CN; and R⁶ is H, halogen, C₁-C₆ alkyl, C₂-C₇ alkenyl, C₂-C₇ alkynyl, —CN, or optionally substituted phenyl. In some further embodiments, R⁵ is H, halogen, or CN.

In some embodiments of the foregoing methods wherein the ERβ selective ligand has Formula VII, A and A′ are each OH; and at least one of R¹ and R² is halogen.

In some embodiments of the foregoing methods wherein the ERβ selective ligand has Formula VII, A and A′ are each OH; and R⁴ and R⁶ are each, independently, halogen, C₂-C₇ alkynyl, or —CN. In some further embodiments, R⁵ is H.

In some embodiments of the foregoing methods, the ERβ selective ligand has the Formula X:

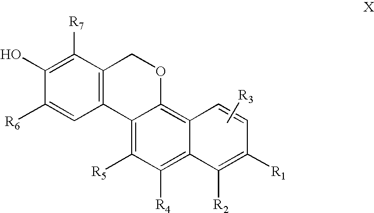

or is a pharmaceutically acceptable salt or prodrug thereof wherein:
R₁ and R₂ are each, independently, selected from hydrogen, hydroxyl, alkyl of 1-6 carbon atoms, alkenyl of 2-6 carbon atoms, alkynyl of 2-7 carbon atoms, alkoxy of 1-6 carbon atoms, or halogen; wherein the alkyl or alkenyl moieties of R₁, or R₂ may be optionally substituted with hydroxyl, —CN, halogen, trifluoroalkyl, trifluoroalkoxy, —NO₂, or phenyl; and provided that at least one of R₁ or R₂ is hydroxyl;
R₃, R₄, R₅, R₆, and R₇ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms each independently selected from O, N or S; wherein the alkyl or alkenyl moieties of R₄, R₅, R₆, or R₇ may be optionally substituted with hydroxyl, —CN, halogen, trifluoroalkyl, trifluoroalkoxy, —NO₂, or phenyl; wherein the phenyl moiety of R₄ or R₅ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl.

In some embodiments of the foregoing methods wherein the ERβ selective ligand has Formula X, R₁ is hydrogen, hydroxyl, or halogen. In some further embodiments, R₁ is hydroxyl.

In some embodiments of the foregoing methods wherein the ERβ selective ligand has Formula X, R₂ is hydrogen, hydroxyl, or halogen.

In some embodiments of the foregoing methods wherein the ERβ selective ligand has Formula X, R₃ is hydrogen, hydroxyl, or halogen.

In some embodiments of the foregoing methods wherein the ERβ selective ligand has Formula X, R₁, R₂, and R₂ are each, independently, selected from hydrogen, hydroxyl, and halogen. In some further embodiments, R₁ is hydroxyl.

In some embodiments of the foregoing methods wherein the ERβ selective ligand has Formula X, R₄ and R₅ are each, independently, hydrogen, alkyl of 1-6 carbon atoms, halogen, alkoxy of 1-6 carbon atoms, alkenyl of 2-7 carbon atoms, or —CN, furyl, or thienyl.

In some embodiments of the foregoing methods wherein the ERβ selective ligand has Formula X, R₄ is other than hydrogen. In some further embodiments, R₄ is alkyl of 1-6 carbon atoms, alkoxy of 1-6 carbon atoms, —CN, or alkenyl of 2-7 carbon atoms. In yet further embodiments, R₄ is —CN or alkenyl of 2-7 carbon atoms. In still further embodiments, R₄ is —CN.

In some embodiments of the foregoing methods wherein the ERβ selective ligand has Formula X, R₆ and R₇ are each, independently, hydrogen or halogen.

In some embodiments of the foregoing methods wherein the ERβ selective ligand has Formula X, R₆ and R₇ are each, independently, hydrogen or halogen; and R₄ and R₅ are each, independently, hydrogen, alkyl of 1-6 carbon atoms, alkoxy of 1-6 carbon atoms, halogen, alkenyl of 2-7 carbon atoms, —CN, furyl, or thienyl. In some further embodiments, R₁, R₂, and R₂ are each, independently, selected from hydrogen, hydroxyl, and halogen. In yet further embodiments, R₄ is other than hydrogen. In still further embodiments, R₁ is hydroxyl and R₄ is —CN or alkenyl of 2-7 carbon atoms.

In some further embodiments of the foregoing methods, the ERβ selective ligand is a compound having the formula:

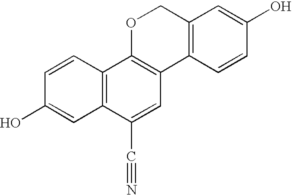

or a pharmaceutically acceptable salt.

The preparation of ERβ selective ligands having Formula VII is described in U.S. patent application Ser. No. 10/846,216, US Published Application US 2005/0009784, published Jan. 13, 2005, and WO 04/103973. The preparation of ERβ selective ligands having Formula X is disclosed in US Published Application US2003/0176491, published Sep. 18, 2003 (U.S. application Ser. No. 10/317163 filed Dec. 11, 2002), U.S. Pat. No. 6,723,747, and PCT US 02/39802, filed Dec. 12, 2002. Each of the foregoing patents and applications is incorporated herein by reference in its entirety.

The present invention also provides compositions comprising a therapeutically effective amount of an ERβ selective ligand, and a traditional mediation for acute lung injuries described herein. In some embodiments, the ERβ selective ligand is applied topically. In some further embodiments, the ERβ selective ligand is non-uterotrophic, non-mammotrophic, or non-uterotrophic and non-mammotrophic.

### Therapeutic Methods

### Methods of Treating or Preventing Acute Lung Injury

The present invention provides methods of treating acute lung injury in a subject in need thereof, comprising administering to the subject a therapeutically effective amount of an ERβ selective ligand or a pharmaceutical composition thereof. The method comprises providing to the subject an effective amount of one or more, preferably one, ERβ selective ligands. In some embodiments, the ERβ selective ligand is administered orally. In some embodiments, the ERβ selective ligand is administered via intravenously injection. In some further embodiments, the ERβ selective ligand is non-uterotrophic, non-mammotrophic, or non-uterotrophic and non-mammotrophic. In some embodiments the subject is a human.

As used herein the terms “treatment”, “treating”, “treat” and the like are refer to obtaining a desired pharmacologic and/or physiologic effect. The effect may be therapeutic in terms of a partial or complete stabilization or cure for a disease and/or adverse effect attributable to the disease. “Treatment” or “treating” as used herein covers any treatment of a disease in a subject, particularly a human, and includes: (a) inhibiting the disease; for example, inhibiting a disease (including one or more symptoms thereof), condition or disorder in an individual who is experiencing or displaying the pathology or symptomatology of the disease, condition or disorder (i.e., arresting further development of the pathology and/or symptomatology; or relieving the disease symptom, i.e., causing regression of the disease or symptom); and (b) ameliorating the disease; for example, ameliorating a disease (including one or more symptoms thereof), condition or disorder in an individual who is experiencing or displaying the pathology or symptomatology of the disease, condition or disorder (i.e., reversing the pathology and/or symptomatology).

As used herein the terms “preventing”, “prevention”, “prevent” and the like are refer to obtaining a desired pharmacologic and/or physiologic effect that may be prophylactic in terms of completely or partially preventing a disease or symptom thereof. “Preventing a disease” or “prevention of a disease” as used herein covers preventing the disease (including one or more symptoms thereof), condition or disorder in an individual who may be predisposed to the disease, condition or disorder but does not yet experience or display the pathology or symptomatology of the disease. In some embodiments, “preventing a disease” further comprises the step of identifying the individual who may be predisposed to the disease, condition or disorder but does not yet experience or display the pathology or symptomatology of the disease. In some embodiments, identifying the individual who may be predisposed to the disease, condition or disorder but does not yet experience or display the pathology or symptomatology of the disease comprises diagnosing the individual.

The terms “individual”, “subject”, “host” and “patient” are used interchangeably and refer to any subject for whom diagnosis, treatment, or therapy is desired, particularly humans. Other subjects may include cattle, dogs, cats, guinea pigs, rabbits, rats, mice, horses, and the like. In some preferred embodiments the subject is a human.

As used herein, “acute lung injury” refers to a critical illness syndrome consisting of acute hypoxemic respiratory failure with bilateral pulmonary infiltrates that are not attributed to left atrial hypertension. “Acute lung injury” also referes to a syndrome of life-threatening progressive pulmonary insufficiency or hypoxemic respiratory failure in the absence of known pulmonary disease (such as emphysema, bronchitis, or chronic obstructive pulmonary disease), usually following a systemic insult such as surgery or major trauma. In some embodiments of the methods of the present invention, the acute lung injury is induced by diseases or disorders other than pulmonary diseases. In some embodiments of the methods of the present invention, the acute lung injury is pulmonary injury caused/induced by an extrapulmonary origin such as neurogenic pulmonary injury, secondary to severe CNS trauma. In some embodiments of the methods of the present invention, the acute lung injury is acute lung injury induced by extrapulmonary diseases. In some embodiments of the methods of the present invention, the acute lung injury is indirect pulmonary injury from trauma, sepsis, and other disorders such as acute pancreatitis, drug overdose. In some embodiments of the methods of the present invention, the acute lung injury is acute lung injury induced by inhalation of noxious fumes, burn, or massive blood transfusion. In some embodiments of the methods of the present invention, the acute lung injury is acute lung injury induced by peritonitis during sepsis, acute lung injury induced by intravenous bacteremia during sepsis, acute lung injury caused by smoke inhalation, acute lung injury occurring in a premature infant with deficiency of surfactant, acute lung injury induced/caused by oxygen toxicity or acute lung injury induced/caused by barotrauma from mechanical ventilation. In some embodiments of the methods of the present invention, the acute lung injury is acute lung injury induced by oxygen toxicity or barotrauma from mechanical ventilation in a premature infant. In some embodiments of the methods of the present invention, the acute lung injury is acute lung injury induced by oxygen toxicity or barotrauma from mechanical ventilation occurring in a premature infant with deficiency of surfactant.

As used herein, the term “bacteremia” refers to the presence of viable bacteria circulating in the blood. Fever, chills, tachycardia, and tachypnea are common acute manifestations of bacteremia. The majority of cases of bacteremia are seen in already hospitalized patients, most of whom have underlying diseases or procedures which render their bloodstreams susceptible to invasion.

As used herein, the term “barotrauma” refers to injury following pressure changes; includes injury to the lung.

As used herein, the terms “administering” or “providing” mean either directly administering the ERβ selective ligand, or administering a prodrug, derivative, or analog of the ERβ selective ligand that will form an effective amount of the ERβ selective ligand within the body. The terms include routes of administration that are systemic (e.g., via injection such as intravenous injection, orally in a tablet, pill, capsule, or other dosage form useful for systemic administration of pharmaceuticals, and the like, such as described herein below), and topical (e.g., creams, solutions, and the like, including solutions such as mouthwashes, for topical oral administration).

The term “in need thereof” and the like as used herein refers to a subject that has been determined to be in need of treatment for a disease such as, for example, acute lung injury, preferably acute lung injury induced by peritonitis during sepsis, acute lung injury induced by intravenous bacteremia during sepsis, acute lung injury caused by smoke inhalation, acute lung injury occurring in a premature infant with deficiency of surfactant, acute lung injury caused by oxygen toxicity or acute lung injury caused by barotrauma from mechanical ventilation. Such a determination may be a result of a medical diagnosis. Further, subjects “in need” of the methods of the present invention include those known or suspected to have been previously diagnosed of acute lung injury, sepsis, severe sepsis or sepsis shock.

ERβ selective ligands are known to those of skilled in the art as compounds which preferentially bind to ERβ relative to ERα. The preparation of certain exemplary ERβ selective ligands, including those of Formulas I and II, such as 2-(3-fluoro-4-hydroxyphenyl)-7-vinyl-1,3-benzoxazol-5-ol (ERB-041), is described in U.S. Pat. No. 6,794,403, and WO 03/050095, each of which is incorporated herein by reference in its entirety. In some embodiments, ERβ selective ligands include compounds set forth in U.S. Pat. No. 6,794,403, WO 03/050095, U.S. patent application Ser. No. 10/316,640, filed Dec. 11, 2002 and published as US 20030181519 on Sep. 25, 2003; U.S. Patent Application Ser. No 60/637,144, filed Dec. 17, 2004, and PCT application no. US2005/045375, each of which is incorporated herein by reference in its entirety.

In some embodiments, the ERβ selective ligand is 2-(3-fluoro-4-hydroxyphenyl)-7-vinyl-1,3-benzoxazol-5-ol, which has the formula:

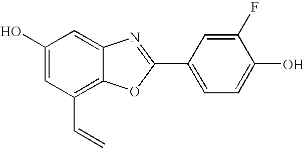

In some embodiments, the ERβ selective ligand is 3-(3-fluoro-4-hydroxyphenyl)-7-hydroxy-1-naphthonitrile, which has the formula:

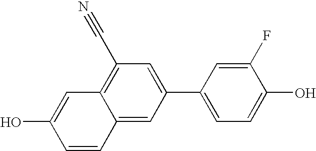

In some embodiments, the ERβ selective ligand is 2,8-dihydroxy-6H-dibenzo[c,h]chromene-12-carbonitrile, which has the formula:

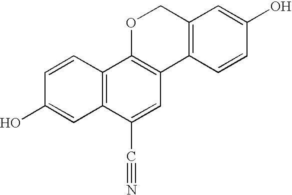

As used herein, the term “ERβ selective ligand” means a compound that preferentially bind to ERβ relative to ERα [i.e., the binding affinity (as measured by IC₅₀) of the ligand to ERβ is greater than its binding affinity to ERα in a standard pharmacological test procedure that measures the binding affinities to ERβ and ERα]. In some preferred embodiments, the binding affinity (as measured by IC₅₀, where the IC₅₀ of 17β-estradiol is not more than 3 fold different between ERα and ERβ) of the ligand to ERβ is at least about 10 times greater than its binding affinity to ERα in a standard pharmacological test procedure that measures the binding affinities to ERβ and ERβ. It is preferred that the ERβ selective ligand will have a binding affinity to ERβ that is at least about 20 times greater than its binding affinity to ERα. It is more preferred that the ERβ selective ligand will have a binding affinity to ERβ that is at least about 50 times greater than its binding affinity to ERα. It is further preferred that the ERβ selective ligand is non-uterotrophic and non-mammotrophic. In some embodiments, the ERβ selective ligands used for the methods of the present invention are ERβ selective agonists. In addition, the binding affinity of an ERβ selective ligand to ERβ receptor is less than about 100 nM, about 50 nM, about 40 nM, about 30 nM, about 20 nM, 10 nM, about 5 nM or about 2 nM. In some embodiments, the binding affinity to ERβ receptor of the ERβ selective ligands described herein is less than about about 50 nM, about 40 nM, about 30 nM, about 20 nM, 10 nM, about 5 nM or about 2 nM.

As used in accordance with this invention, the term “non-uterotrophic” means producing an increase in wet uterine weight in a standard pharmacological test procedure of less than about 50% of the uterine weight increase observed for a maximally efficacious dose of a positive control in the same procedure. In some preferred embodiments the standard pharmacological test procedure measuring uterotrophic activity is the pharmacological test procedure published in Harris H A, et al, Endocrinology 2002;143(11):4172-4177, referred to hereinafter as the “uterotrophic test procedure”. In some embodiments the positive control is 17β-estradiol, 17α-ethinyl-17β-estradiol or diethylstilbestrol (DES). It is preferred that the increase in wet uterine weight will be less than about 25% of that observed for the positive control, and more preferred that the increase in wet uterine weight will be less than about 10% of that observed for the positive control. It is most preferred that the non-uterotrophic ERβ selective ligand will not significantly increase wet uterine weight (p>0.05), as determined by analysis of variance using a least significant difference test, compared with a control that is devoid of uterotrophic activity (e.g. vehicle). The maximally efficacious dose of the positive control will vary depending on a number of factors including but not limited to the specific assay methodology, the identity of the positive control, amount and identity of vehicle, etc. In some embodiments, the positive control is 17β-estradiol and the maximally efficacious dose is between 0.1 μg/kg and 100 μg/kg, preferably between 1.0 μg/kg and 30 μg/kg; more preferably between 3 μg/kg and 30 μg/kg; and more preferably between 10 μg/kg and 20 μg/kg. In some embodiments, the positive control is 17α-ethinyl-17β-estradiol and the maximally efficacious dose is between 0.1 μg/kg and 100 μg/kg, preferably between 1.0 μg/kg and 30 μg/kg; more preferably between 3 μg/kg and 30 μg/kg; and more preferably between 10 μg/kg and 20 μg/kg. In some embodiments, the positive control is DES and the maximally efficacious dose is between 0.1 μg/kg and 100 μg/kg, preferably between 1.0 μg/kg and 30 μg/kg; more preferably between 3 μg/kg and 30 μg/kg; and more preferably between 10 μg/kg and 20 μg/kg.

As used herein, the term “non-mammotrophic” means a compound that does not stimulate mammary gland development. In some embodiments, “non-mammotrophic” refers to producing an increase in defensin β1 mRNA in a standard pharmacological test procedure of less than about 50% of the defensin β1 mRNA increase observed for a maximally efficacious dose of 17β-estradiol (given in combination with progesterone) in the same procedure. In some embodiments, the standard pharmacological test procedure measuring mammotrophic activity is the Mammary End Bud Test Procedure. In some embodiments it is preferred that the increase defensin β1 mRNA will be less than about 25% of that observed for a positive control, and more preferred that the increase in defensin β1 mRNA will be less than about 10% of that observed for the positive control. It is most preferred that the non-mammotrophic ERβ selective ligand will not significantly increase defensin β1 mRNA (p>0.05) compared with a control that is devoid of mammotrophic activity (e.g. vehicle). In some embodiments, “non-mammotrophic” compounds can be identified using assays for measuring defensin β1 levels including, but not limited to, RT-PCR, Northern blots, in situ hybridization, immunohistochemistry (IHC), and Western blots. In some embodiments, compounds that are “non-mammotrophic” can be determined using histology, e.g., by confirming the absence of physical markers of mammary gland development. In some embodiments, indicators include without limitation, ductal elongation and appearance of lobulo-alveolar endbuds.

The present invention also provides methods of preventing acute lung injury in the subject who is suspected of being at risk for acute lung injury. In some embodiments, the method of the present invention further comprises identifying the subject who is suspected of being at risk for acute lung injury. In some further embodiments, identifying the subject who is suspected of being at risk for acute lung injury comprises diagnosing the subject. In some embodiments, the subject suspected of being at risk for acute lung injury is selected from a subject being suspected of being at risk for sepsis, severe sepsis or septic shock, and a premature infant with deficiency of surfactant. In some embodiments, the subject suspected of being at risk for acute lung injury is selected from a subject who has been previously diagnosed of sepsis, severe sepsis, or septic shock. In some embodiments, the subject suspected of being at risk for acute lung injury is a premature infant. In some embodiments, the subject suspected of being at risk for acute lung injury is a premature infant subject to supplemental oxygen, assisted ventilation, or supplemental oxygen and assisted ventilation. In some embodiments, the subject suspected of being at risk for acute lung injury is a premature infant with deficiency of surfactant. In some embodiments, the subject suspected of being at risk for acute lung injury is a premature infant with deficiency of surfactant who is subject to supplemental oxygen, assisted ventilation, or supplemental oxygen and assisted ventilation. In some embodiments, the subject suspected of being at risk for acute lung injury is selected from a subject being suspected of being at risk for inhalation of noxious fumes, burn, massive blood transfusion, acute pancreatitis, or drug overdose. In some embodiments, the subject suspected of being at risk for acute lung injury is selected from a subject being suspected of being at risk for inhalation of noxious fumes such as smoke in a fire.

### Methods of Treating or Preventing Symptoms of Acute Lung Injuries

The present invention further provides methods of treating at least one symptom of acute lung injuries. The methods comprise providing to the subject an effective amount of an ERβ selective ligand a pharmaceutical composition thereof. In some embodiments, the at least one symptom is selected from lung hemorrhage, and hyaline membrane formation. In some embodiments, the at least one symptom is selected from pulmonary infiltrates. In some embodiments, the at least one symptom is selected from increased respiratory rate. In some embodiments, the at least one symptom is selected from lung edema and lung inflammation. In some embodiments, the at least one symptom is selected from increased perivascular fluid flux, increased transvascular fluid flux, prevalent interstitial edema and alveolar collapse. In some embodiments, the at least one symptom is selected from prevalent interstitial edema and alveolar collapse. In some embodiments, the ERβ selective ligand is administered orally. In some embodiments, the ERβ selective ligand is administered intravenously. In some embodiments, the ERβ selective ligand is administered via injection such as intravenous injection. In some further embodiments, the ERβ selective ligand is non-uterotrophic, non-mammotrophic, or non-uterotrophic and non-mammotrophic.

The present invention also provides methods of preventing at least one symptom of acute lung injuries in a subject who is suspected of being at risk for acute lung injury. The methods comprise providing to the subject an effective amount of an ERβ selective ligand a pharmaceutical composition thereof. In some further embodiments, the methods comprise identifying the subject who is suspected of being at risk for acute lung injury. In some further embodiments, identifying the subject who is suspected of being at risk for acute lung injury comprises diagnosing the subject. In some further embodiments, identifying the subject who is suspected of being at risk for acute lung injury comprise diagnoses. In some further embodiments, the subject suspected of being at risk for acute lung injury is selected from a subject being suspected of being at risk for sepsis, a subject being suspected of being at risk for severe sepsis, a subject being suspected of being at risk for septic shock, a premature infant with deficiency of surfactant, a subject being suspected of being at risk for inhalation of noxious fumes, a subject being suspected of being at risk for burn, a subject being suspected of being at risk for massive blood transfusion, a subject being suspected of being at risk for acute pancreatitis, and a subject being suspected of being at risk for drug overdose. In some further embodiments, the at least one symptom is selected from lung hemorrhage, hyaline membrane formation, pulmonary infiltrates, lung edema, lung inflammation, increased perivascular fluid flux, increased transvascular fluid flux, prevalent interstitial edema, alveolar collapse and increased respiratory rate.

As used herein, the term “alkyl” is meant to refer to a saturated hydrocarbon group which is straight-chained or branched. Example alkyl groups include methyl (Me), ethyl (Et), propyl (e.g., n-propyl and isopropyl), butyl (e.g., n-butyl, isobutyl, s-butyl, t-butyl), pentyl (e.g., n-pentyl, isopentyl, neopentyl) and the like. Alkyl groups can contain from 1 to about 20, 1 to about 10, 1 to about 8, 1 to about 6, 1 to about 4, or 1 to about 3 carbon atoms. In some embodiments, alkyl groups can be substituted with up to four substituent groups, as described below. As used herein, the term “lower alkyl” is intended to mean alkyl groups having up to six carbon atoms.

As used herein, “alkenyl” refers to an alkyl group having one or more double carbon-carbon bonds. An alkenyl group can contain from 2 to about 20, from 2 to about 10, from 2 to about 8, from 2 to about 6, from 2 to about 4, or from 2 to about 3 carbon atoms. Example alkenyl groups include ethenyl, propenyl, butenyl, pentenyl, hexenyl, butadienyl, pentadienyl, hexadienyl, and the like. In some embodiments, alkenyl groups can be substituted with up to four substituent groups, as described below.

As used herein, “alkynyl” refers to an alkyl group having one or more triple carbon-carbon bonds. An alkynyl group can contain from 2 to about 20, from 2 to about 10, from 2 to about 8, from 2 to about 6, from 2 to about 4, or from 2 to about 3 carbon atoms. Examples of alkynyl groups include ethynyl, propynyl, butynyl, pentynyl, and the like. In some embodiments, alkynyl groups can be substituted with up to four substituent groups, as described below.

As used herein, “cycloalkyl” refers to non-aromatic carbocyclic groups including cyclized alkyl, alkenyl, and alkynyl groups. Cycloalkyl groups can be monocyclic (e.g., cyclohexyl) or poly-cyclic (e.g. 2, 3, or 4 fused ring, bridged, or spiro monovalent saturated hydrocarbon moiety), wherein the carbon atoms are located inside or outside of the ring system. A cycloalkyl group can have from 3 to about 20 carbon atoms, 3 to about 14 carbon atoms, 3 to about 10 carbon atoms, 3 to 7 carbon atoms, 3 to about 6 carbon atoms, 3 to about 5 carbon atoms, 3 to 4 carbon atoms, or 4 to about 7 carbon atoms. Cycloalkyl groups can further have 0, 1, 2, or 3 double bonds and/or 0, 1, or 2 triple bonds. Any suitable ring position of the cycloalkyl moiety may be covalently linked to the defined chemical structure. Examples of cycloalkyl groups include cyclopropyl, cyclopropylmethyl, cyclobutyl, cyclopentyl, cyclohexyl, cyclohexylmethyl, cyclohexylethyl, cycloheptyl, cyclopentenyl, cyclohexenyl, cyclohexadienyl, cycloheptatrienyl, norbornyl, norpinyl, norcarnyl, adamantyl, spiro[4.5]deanyl, homologs, isomers, and the like. Also included in the definition of cycloalkyl are moieties that have one or more aromatic rings fused (i.e., having a bond in common with) to the cycloalkyl ring, for example, benzo derivatives of cyclopentane(indanyl), cyclohexane(tetrahydronaphthyl), and the like. In some embodiments, cycloalkyl groups can be substituted with up to four substituent groups, as described below.

As used herein, “hydroxy” or “hydroxyl” refers to OH.

As used herein, “halo” or “halogen” includes fluoro, chloro, bromo, and iodo.

As used herein, “cyano” refers to CN.

As used herein, “alkoxy” refers to an —O-alkyl group. Example alkoxy groups include methoxy, ethoxy, propoxy (e.g., n-propoxy and isopropoxy), t-butoxy, and the like. An alkoxy group can contain from 1 to about 20, 1 to about 10, 1 to about 8, 1 to about 6, 1 to about 4, or 1 to about 3 carbon atoms. In some embodiments, alkoxy groups can be substituted with up to four substituent groups, as described below.

As used herein, the term “perfluoroalkoxy” indicates a group of formula —O-perfluoroalkyl.

As used herein, “haloalkyl” refers to an alkyl group having one or more halogen substituents. Examples of haloalkyl groups include CF₃, C₂F₅, CHF₂, CCl₃, CHCl₂, C₂Cl₅, and the like. An alkyl group in which all of the hydrogen atoms are replaced with halogen atoms can be referred to as “perhaloalkyl.” Examples perhaloalkyl groups include CF₃ and C₂F₅.

As used herein, “haloalkoxy” refers to an —O-haloalkyl group.

As used herein, “aryl” refers to aromatic carbocyclic groups including monocyclic or polycyclic aromatic hydrocarbons such as, for example, phenyl, 1-naphthyl, 2-naphthyl anthracenyl, phenanthrenyl, and the like. In some embodiments, aryl groups have from 6 to about 20 carbon atoms or from 6 to about 10 carbon atoms. In some embodiments, aryl groups can be substituted with up to four substituent groups, as described below.

As used herein, “heterocyclic ring” is intended to refer to a monocyclic aromatic or non-aromatic ring system having from 5 to 10 ring atoms and containing 1-3 hetero ring atoms each independently selected from O, N and S. In some embodiments, one or more ring nitrogen atoms can bear a substituent as described herein. In some embodiments, one or more ring carbon atoms can bear a substituent as described herein. In some embodiments, heterocyclic ring groups can be substituted with up to four substituent groups, as described below. Examples of 5-6 membered heterocyclic rings include furan, thiophene, pyrrole, isopyrrole, pyrazole, imidazole, triazole, dithiole, oxathiole, isoxazole, oxazole, thiazole, isothiazolem oxadiazole, furazan, oxatriazole, dioxazole, oxathiazole, tetrazole, pyran, pyridine, pyridazine, pyrimidine, pyrazine, triazine, oxazine, oxathiazine, and oxadiazine. In some ebodiments, examples of heterocyclic ring include furan, thiophene, and thiazole.

As used herein, “arylalkyl” or “aralkyl” refers to a group of formula—alkyl-aryl. Preferably, the alkyl portion of the arylalkyl group is a lower alkyl group, i.e., a C₁₋₆ alkyl group, more preferably a C₁₋₃ alkyl group. The aryl portion of the arylakyl group can have have from 6 to about 20 carbon atoms or from 6 to about 10 carbon atoms. Examples of aralkyl groups include benzyl and naphthylmethyl groups. In some embodiments, arylalkyl groups can be substituted with up to four substituent groups, as described below.

Examples of suitable substituent groups (for alkyl, alkenyl, alkynyl, alkoxy, heterocyclic ring, cycloalkyl, aryl, and arylalkyl) include hydroxyl, CN, halogen, trifluoroalkyl, trifluoroalkoxy, NO₂, phenyl, optionally substituted phenyl, alkyl of 1-6 carbon atoms, alkenyl of 2-7 carbon atoms, alkynyl of 2-7 carbon atoms, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, benzoy, —CHO, carboxy, acyl, trialkylsilyl, and optionally substituted phenyl. Examples of optionally substituted phenyl include phenyl optionally substituted by 1, 2, 3, 4 or 5 substituents each independently selected from alkyl of 1-6 carbon atoms, alkenyl of 2-7 carbon atoms, alkynyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, and benzoyl. In some embodiments, examples of substituent groups for alkyl or alkenyl include hydroxyl, alkoxy of 1-6 carbon atoms, —CN, halogen, trifluoroalkyl, trifluoroalkoxy, NO₂, and phenyl. In some embodiments, examples of substituent groups for aryl, arylalkyl, cycloalkyl, or heterocyclic ring include alkyl of 1-6 carbon atoms, alkenyl of 2-7 carbon atoms, alkynyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, and benzoyl. In some embodiments, examples of substituent groups for alkenyl or alkynyl include halogen, hydroxyl, alkoxy of 1-6 carbon atoms, —CN, —CHO, acyl, trifluoroalkyl, trialkylsilyl, and optionally substituted phenyl.

At various places in the present specification substituents of compounds of the invention are disclosed in groups or in ranges. It is specifically intended that the invention include each and every individual subcombination of the members of such groups and ranges. For example, the term “C₁₋₆ alkyl” or “alkyl of 1-6 carbon atoms” is specifically intended to individually disclose methyl, ethyl, propyl, isopropyl, n-butyl, sec-butyl, isobutyl, etc.

### Administration and Pharmaceutical Compositions

The ERβ selective ligand agonist may be administered alone or may be delivered in a mixture with other drugs, such as recombinant human activated protein C[Maybauer, M. O., et al., “recombinant human activated protein C improves pulmonary function in ovine acute lung injury resulting from smoke inhalation and sepsis”; Crit. Care Med., 2006, Vol. 34, No. 9, pages 2432-38], for treating acute lung injuries. In some embodiments, a common administration vehicle (e.g., pill, tablet, implant, injectable solution, etc.) would contain both an ERβ selective ligand and additional therapeutic agent(s). Thus, the present invention also provides pharmaceutical compositions, for medical use, which comprise the ERβ selective ligand of the invention together with one or more pharmaceutically acceptable carriers thereof and optionally other therapeutic ingredients.

In accordance with the present invention, treatment can also include combination therapy. As used herein “combination therapy” means that the patient in need of treatment is treated or given another drug or treatment modality for the disease in conjunction with the ERβ selective ligand of the present invention. This combination therapy can be sequential therapy where the patient is treated first with one and then the other, or the two or more treatment modalities are given simultaneously. Preferably, the treatment modalities administered in combination with the ERβ selective ligands do not interfere with the therapeutic activity of the ERβ selective ligand.

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. It is projected that effective administration of the compounds of this invention may be given at a daily oral dose of from about 5 μg/kg to about 100 mg/kg. The projected daily dosages are expected to vary with route of administration, and the nature of the compound administered. In some embodiments the methods of the present invention comprise administering to the subject escalating doses of an ERβ selective ligand. In some embodiments, the ERβ selective ligand is administered orally. In some embodiments, the ERβ selective ligand is administered via injection such as intravenous injection. In some further embodiments, the ERβ selective ligand is non-uterotrophic, non-mammotrophic, or non-uterotrophic and non-mammotrophic.

Such doses may be administered in any manner useful in directing the active compounds herein to the recipient's bloodstream, including orally, via implants, parenterally (including intravenous, intraperitoneal and subcutaneous injections), intraarticularly, rectally, intranasally, intraocularly, vaginally, or transdermally.

Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time-release formulations to alter the absorption of the active compound(s). The oral formulation may also consist of administering the active ingredient in water or a fruit juice, containing appropriate solubilizers or emulsifiers as needed.

In some cases it may be desirable to administer the compounds directly to the airways in the form of an aerosol.

The compounds of this invention may also be administered parenterally (such as directly into the joint space) or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

In some embodiments, the methods of the invention are performed via intravenous administration (e.g., intravenous injection) of the ERβ selective ligand. Compositions containing the ERβ selective ligands suitable for intravenous administration can be selected, for example, from aqueous pharmaceutical compositions containing ERβ selective ligands described in U.S. Patent Application Ser. No 60/773,028, filed Feb. 14, 2006, incorporated herein by reference in its entirety. In some embodiments, it is advantageous to administer the ERβ selective ligand via intravenous injection especially when oral administration is difficult or not practical for the subject.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

Additional numerous various excipients, dosage forms, dispersing agents and the like that are suitable for use in connection with the solid dispersions of the invention are known in the art and described in, for example, Remington's Pharmaceutical Sciences, 17th ed., Mack Publishing Company, Easton, Pa., 1985, which is incorporated herein by reference in its entirety.

### Kits

In some embodiments, a kit comprising one or more ERβ selective ligands useful for the treatment of the diseases or disorders described herein is provided. In some further embodiments, the kit comprises one or more ERβ selective ligands useful for the treatment of the diseases or disorders described herein, and instructions comprising a direction how to administer such ERβ selective ligands for the treatment of the diseases or disorders described herein. In some embodiments, the kit comprises a container and a label or package insert on or associated with the container. Suitable containers include, for example, bottles, vials, syringes, etc. The containers can be formed from a variety of materials such as glass or plastic. The container holds or contains a composition that is effective for treating the disease or disorder of choice and may have a sterile access port (for example the container may be an intravenous solution bag or a vial having a stopper pierceable by a hypodermic injection needle). At least one active agent in the composition is an ERβ selective ligand. The label or package insert indicates that the composition is used for treating a patient having or predisposed to acute lung injuries, such as acute lung injury induced by peritonitis during sepsis, acute lung injury induced by intravenous bacteremia during sepsis, acute lung injury caused by smoke inhalation, acute lung injury occurring in a premature infant with deficiency of surfactant, acute lung injury caused by oxygen toxicity or acute lung injury caused by barotrauma from mechanical ventilation. The article of manufacture can further include a second container having a pharmaceutically acceptable diluent buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes. Optionally the kit may contain other components including, without limitations, traditional medicaments for the treatment of the diseases or disorders described herein. ERβ selective ligands can be tested using a number of methods known to those skilled in the art. Such methods include, for example, measuring relative binding affinities to ERβ and ERα and assessing on ore more activities in well-known assays.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

### Evaluation of Binding Affinities to ERβ and ERα

Compounds can be evaluated for their ability to compete with 17β-estradiol using both ERβ and ERα. This test procedure provides the methodology for one to determine the relative binding affinities for the ERβ or ERα. The procedure used is as described in Harris H A, et al, Steroids 2002;67(5):379-384.

### Example 2

### Evaluation of Uterotrophic Activity

Uterotrophic activity of a test compound can be measured according to the standard pharmacological test procedure as published in Harris H A, et al, Endocrinology 2002;143(11):4172-4177. For the sake of brevity, the standard pharmacological test procedure as published in Harris et al. will be referred to as the “uterotrophic test procedure”.

### Example 3

### Evaluation in the Mammary End Bud Test Procedure

Estrogens are required for full ductal elongation and branching of the mammary ducts, and the subsequent development of lobulo-alveolar end buds under the influence of progesterone. In this test procedure, the mammotrophic activity of ERβ selective compounds can be evaluated as follows. Seven week old C57/bl6 mice (Taconic Farms, Germantown, N.Y.) are ovariectomized and rested for about nine days. Animals are housed under a 12-hour light/dark cycle and fed a casein-based Purina Laboratory Rodent Diet 5K96 (Purina, Richmond, Ind.) and water ad libidtum. Mice are then dosed for seven days with vehicle, 17β-estradiol (1 μg/kg, subcutaneously in a vehicle of 50% DMSO/50% 1× Dulbecco's phosphate buffered saline) or an ERβ selective ligand (various doses, orally in a vehicle of 2% Tween-80/0.5% methylcellulose). For the final four days, mice are also dosed subcutaneously with progesterone (30 mg/kg, subcutaneously in a vehicle of 50% DMSO/50% 1× Dulbecco's phosphate buffered saline). On the seventh day, mice are euthanized and the number 4 or 9 inguinal mammary gland and underlying fat pad are excised. The fat pad is analyzed for defensin 1β mRNA expression as a marker of end bud proliferation. Total RNA is prepared individually from each mammary gland. Each sample is homogenized in 2 mLs of QIAzol lysis reagent (Qiagen; Valencia, Calif.) for 15-25 seconds using a Polytron homogenizer PT3100 (Brinkmann; Westbury, N.Y.). After 1 mL of this homogenate is extracted with 0.2 mL of chloroform and centrifuged at 4° C. for 15 minutes, about 0.5 mL aqueous phase is collected. The RNA from the aqueous phase is then purified using Qiagen RNeasy kits according to the manufacturer's protocol. The trace genomic DNA in RNA sample is removed by on column RNase-Free DNase treatment during RNA purification. The RNA concentration is adjusted to 0.05 mg/ml for assay. Messenger RNA expression is analyzed using real-time quantitative-PCR on an ABI PRISM 7700 Sequence Detection System according to the manufacturer's protocol (Applied Biosystems Inc; Foster City Calif.).

Defensin β1 sequences are known to the art skilled and include, for example, GenBank accession numbers BC024380 (mouse) and NM_{—}005218 (human). The sequences of primers and labeled probes used for defensin β1 mRNA detection are as follows: forward primer, 5′-AATGCCTTCAACATGGAGGATT-3 (SEQ ID NO:1); reverse primer, 5′-TTACAGGTTCCCTGTAGTTTGGTATTAG-3′ (SEQ ID NO:2); probe, 5′FAM-TGTCTCCGCTCCAGCTGCCCA-TAMRA-3′ (SEQ ID NO:3). To compare mRNA expression levels between samples, defensin β1 mRNA expression is normalized to 18S RNA expression using primers and labeled probes from an Applied Biosystems TaqMan ribosomal RNA control reagent kit (VIC probe) for 18S mRNA detection. The expected result is that defensin β1 mRNA will be strongly upregulated by the combination of 17β-estradiol and progesterone, but not by either compound given alone. Test compounds, then, are evaluated for their ability to substitute for 17β-estradiol in this regimen.

### Example 4

### Preparation of 100 mL of an Aqueous Formulation Containinglo mg/mL of Compound 1in 15% Hydroxypropyl-beta-cyclodextrin (HPBCD)/0.06N NaOH pH 9.1

1. 1.0 g of 3-(3-fluoro-4-hydroxy-phenyl)-7-hydroxy-naphthalene-1-carbonitrile (Compound 1) was weighed into a tared container.
2. 15.00 g of HPBCD was weighed out and transfer to the container.
3. 82.35 g Sterile Water for Injection was added to the container.
4. 6.25 g (6 mL) of 1N NaOH was added to the container.
5. The contents of the container were mixed by continuous stirring to dissolve the solids. Up to 30 minutes may be required to completely dissolve the Compound 1.
6. When dissolution was complete, the pH was confirmed to be ˜9.0-9.3.
7. The solution was then filtered through a Millipore Millex-GV 0.22 u PVDF filter.
8. The final pH was then reconfirmed to be 9.1.

The composition of the Formulation is shown below in Table 1.

TABLE 1

| | Percent | | |
|---|---|---|---|
| | Composition | | |
| Ingredient | (w/v) | Quantity in 100 mL | |
| Compound 1 | 1.00 | 1.00 | g |
| Hydroxypropyl-beta-cyclodextrin | 15.00 | 15.00 | g |
| NaOH 1N | 6.25 | 6.25 | g (= 6 mL) |
| Sterile water for Injection | qs | 82.35 | g |
| Total | | 104.6 | g = 100 mL |

The density of the final solution was 1.046 g/mL

### Example 5

### Evaluation of An Estrogen Receptor-β Selective Agonist (Compound 1) in Murine Cecal Ligation and Puncture (CLP) Model of Polymicrobial Sepsis

Murine cecal ligation and puncture is an accepted model of sepsis and was performed according to a previously published protocol [Opal et al., “evaluation of the safety of recombinant P-selectin glycoprotein ligand-immunoglobulin G fusion protein in experimental models of localized and systemic infection,” Shock 2001;15:285-90]. Compound 1, when administered beginning at the time CLP induction, provides a survival advantage in this model [Cristofaro et al. Critical Care Medicine 2006; 34:2188-2193]. Acute lung injury is a documented component of this peritonitis-induced sepsis, and the model has been used to study the effects of other pharmaceutical agents on acute lung injury in sepsis [Tsujimoto et al. Shock 2005;23:39-44; and Singleton et al. Am J Physiol Regul Integr Comp Physiol (Jan. 18, 2007)].

Mice were euthanized at 48 hours following CLP and treatment with vehicle or Compound 1, both treatments having begun at the time of CLP.

Compound 1 was given at a range of doses orally at time 0, 24 and 48 hours following CLP in male and female BALB/c mice Survival, inflammatory markers, lung histopathology, and microbiologic parameters were assessed.

Multiple lung specimens were taken at necropsy from 8 vehicle- and 8 Compound 1—treated animals and evaluated by an independent pathologist unaware of the treatment group assignments. Treatment with Compound 1 reduced lung lesions (such as lung edema and lung inflammation) comparing to that with vehicle (Compound 1: 1.0±0.76 vs. vehicle: 3.08±0.74, p<0.001). A standard scoring scheme was used: 0: normal, 1: mild edema, inflammation, 2: moderate inflammatory, 3: marked segmental, 4: marked diffuse inflammation and damage.

### Example 5a

### Lung Tissue Gene Expression in the mCLP Model

In order to further define the activity of Compound 1 in murine CLP, satellite groups of mice were treated intravenously at the time of CLP induction surgery with vehicle or Compound 1. An additional group of mice(surgical sham group) were subjected to the anesthesia and laparotomy, the cecum was manipulated but not ligated, and then the abdomen was closed. Because mCLP produces a progressive sepsis state, the animals become extremely ill and begin to succumb between 48-72 hours after mCLP. Thus, these animals were euthanized at 48 hours and lung tissue samples collected for gene expression analysis.

Messenger RNA was prepared by standard techniques, and the samples were processed on the Mouse430_{—}2 Affymetrix commercial array, containing 45,037 non-control probe sets. Probe sets that were called present by the Affymetrix detection algorithm for at least one sample of any cohort and also had an average normalized Affymetrix signal value greater than fifty for the same cohort (robust probe sets).

### A. Probe Sets Correlated With Onset of the CLP-Induced Sepsis

Probe sets that significantly changed as a result of the CLP-induced sepsis model when compared to sham-operated animals, were derived via t-test. The IV cohorts produced a set of 3747 probe sets that are differentially expressed at a significant level (p<0.05). To identify the set of probe sets correlated with onset of the CLP-induced sepsis, the two groups were intersected such that only significant probe sets common to both analysis sets with the same fold change direction remained. On the list of 369 resulting probe sets using Ingenuity Pathways Analysis (IPA), 211 of the 369 probe sets were eligible for generating pathways. This analysis showed that three pathways were significantly overrepresented in the data. These pathways are identified in Table 2.

TABLE 2
Three pathways that were significantly overrepresented in the CLP
model of sepsis by Ingenuity Pathways Analysis (IPA) based on 211
of 369 significantly regulated probe sets in lung tissue.

| Pathway | Significance (p-value) |
|---|---|
| IL-10 Signaling | 2.73E−03 |
| Complement and | 5.99E−03 |
| Coagulation Cascades | |
| Sulfur Metabolism | 6.12E−03 |

Additionally, IPA showed that a number of functions were overrepresented in the data. These include cell death, cell cycle, neurological disease, inflammatory disease, immune response, hematological disease, and cancer among others.

TABLE 3
Probe sets common to a CLP-induced sepsis model in lung tissues.

| Pathway | Significance (p-value) |
|---|---|
| JAK/Stat Signaling | 2.67E−03 |
| IL-10 Signaling | 4.19E−03 |
| PI3K/AKT Signaling | 9.36E−03 |
| NF-kB Signaling | 1.34E−02 |

B. Genes Correlated with Activity of Compound 1 Treatment in the CLP-Induced Sepsis Model

Probe sets that are significantly responsive to Compound 1 treatment in the CLP-induced model of sepsis are those that are first significantly responsive in the CLP-induced sepsis model when compared to sham-operated animals and are also significantly responsive to Compound 1 treatment when compared to the CLP untreated animals such that the Compound 1—treated profile approaches the sham-operated profile. A heat map of differential gene expression was used to detect those gene transcripts with gene expression decreasing and those gene transcripts with gene expression increasing.

Using Ingenuity Pathways Analysis (IPA), 277 of the 522 probe sets were eligible for generating pathways. This analysis showed that seven pathways were significantly overrepresented in the data. These pathways are identified in Table 4.

TABLE 4
Seven pathways that are significantly overrepresented in the Compound 1
treatment of the CLP-induced sepsis model by Ingenuity Pathways
Analysis (IPA) based on 277 of 522 significantly regulated probe sets
in lung tissue.

| Pathway | Significance (p-value) |
|---|---|
| Sterol Biosynthesis | 1.17E−02 |
| p38 MAPK Signaling | 1.92E−02 |
| Hypoxia Signaling in | 2.53E−02 |
| the Cardiovascular | |
| System | |
| B Cell Receptor | 2.70E−02 |
| Signaling | |
| SAPK/JNK Signaling | 3.45E−02 |
| ERK/MAPK Signaling | 3.58E−02 |
| Death Receptor | 4.07E−02 |
| Signaling | |

In Summary, Compound 1 significantly decreased multiple proinflammatory pathways in the lungs of mice subjected to CLP. The decrease in these gene transcripts, that have been related to acute lung injury, is consistent with the decrease in histologic lesions and increased survival seen in Compound 1—treated animals.

### Example 6

### Evaluation of an Estrogen Receptor-β Selective Agonist (Compound 1) in an Intravenous E. Coli Challenge Model in Baboons

The intravenous E. coli infusion model of sepsis in baboons has been used for many years in sepsis research [Taylor F B Crit Care Med 2001 ;29:S78-89], and it has acute lung injury as one of its pathophysiological features [Sabharwal A K et al. Am J Resp Crit Care 1995; 151:758-67].

Compound 1 was given at a range of doses at time 0, 24 and 48 hours intravenously at a dose of 10 mg/kg in baboons 5 minutes before and then at 2, 24, 48, 72 and 96 hours following E. coli challenge. Survival, inflammatory markers, lung histopathology, and microbiologic parameters were assessed.

In the baboon study the lungs were examined at euthanasia of the animals when moribund or at the end of the 7 day experiment. Animals were assigned to vehicle and Compound 1 E. coli challenge. The lung specimens were evaluated by an independent pathologist unaware of treatment group assignments. Compound 1 attenuated clinical signs of pulmonary injury in the baboon model as measured by the percent change in respiratory rate from baseline values, and histopathologic examination revealed decreased pulmonary damage as evidenced by decreased intrapulmonary hemorrhage and absence of hyaline membrane formation in the compound 1 treated animals.

### Example 6a

### Plasma Proteome and Gene Transcription of Peripheral Blood Mononuclear Cells Analysis in the IV Live E. Coli Challenge Model in Baboon

The plasma proteome and gene transcription of peripheral blood mononuclear cells (PBMC) were investigated using the baboon model of sepsis to evaluate the effect of Compound 1 on progression of the disease. The study comprised three treatment groups—sham-treated, vehicle, and Compound 1—of three baboons each. Blood samples were drawn at multiple time points (0, 0.5, 1, 2, 3, 4, 6, 24, 48, and 168 hours) from each baboon. NuGen amplified RNA was prepared from peripheral blood mononuclear cells (PBMC) isolated from the 0, 1, 6, and 24 hour blood samples. RNA levels were then measured using Affymetrix rhesus monkey Genechips, which interrogated 52,865 transcripts. Of interest was whether expression levels of one or more of these transcripts differed among the three treatment groups over the course of the study.

### A. Statistical Methods

Genechip data were processed using Affymetrix MAS 5 software to calculate detection p-values and signal (expression) values. Detection p-values were used to generate Affymetrix Absent, Marginal, or Present calls (p-value≧0.065: Absent; 0.05≦p-value<0.065: Marginal; p-value<0.05: Present). Signal values were normalized by the standard MAS 5 procedure.

An initial nominal filtering of the transcripts was done to eliminate those for which none of the samples had a Present call. This filter reduced the number of transcripts for further analysis to 43,181.

To adjust for differences among animals in baseline expression values, change from baseline expression was analyzed. Change from baseline expression values were calculated using log2-transformed signal values. For each transcript, the log2 signal value for an animal at the 0 hour (baseline) sampling time was subtracted from the log2 signal value for the same animal for each later sampling time to yield change from baseline values for 1, 6, and 24 hours for that animal.

Repeated-measures analysis of variance (ANOVA) was used to compare (log2-transformed) change from baseline expression values in the treatment groups across the sampling time points. The ANOVA model included terms for treatment group and sampling time, and a term for the interaction between treatment group and time. The model also included a compound symmetry covariance parameter to account for any within-animal correlation between measurements at different sampling times from the same animal. Separate ANOVAs were run for each transcript. Pairwise comparisons between treatment groups were computed for mean concentrations across all times as well as for each time point separated. These pairwise comparisons were based on two-sided t-tests, with the error term for the t-tests based on the overall error term from the ANOVA.

Due to the substantial number of statistical tests performed, false discovery rates (FDRs) were used to adjust the raw p-values from F-tests and t-tests for multiple comparisons. The FDRs were computed to control the family-wise error level by calculating FDRs across transcripts separately for each set of comparisons (e.g., each pairwise comparison at a particular sampling time).

### B. Results

The ANOVAs provided evidence of statistically relevant differences among treatment groups in PBMC expression levels for a large number of transcripts. Statistical relevance can be judged using the raw p-values obtained from the ANOVA and/or pairwise comparison tests, or by using FDRs, which adjust for the fact that a large number of statistical tests were performed. If raw p-values are used, a relatively stringent criterion, such at p<0.001, should be employed to compensate for the fact that a large number of tests were performed.

Additional statistical “protection” could be provided by first subsetting to only those transcripts that have a small p-value or FDR for the “treatment-by-time” interaction F-test in the ANOVA. Using a raw p-value criterion of <0.001, there are 1130 such transcripts; an FDR criterion of <0.05 yields 1538 transcripts.

Three gene transcripts, related to acute lung injury were detected in the PBMCs derived from the peripheral blood. They included: early growth response 3 (EGR-3), Nuclear receptor subfamily 4, group A, member 3 (NR4A3), Hypoxia inducible factor-2 Alpha (HIF2-ALPHA). The first two are regulated by Vascular Endothelial Growth Factor (VEGF), and HIF-Alpha regulates VEGF. All 3 rose from 1 to 6 hours after E coli infusion, but compound 1 treatment decreased all 3 by 24 hours when compared to vehicle treated animals. The VEGF and HIF pathways are activated in acute lung injury and are thought to be protective compensatory responses. Given the reduction in lung injury produced by compound 1 as evidenced by decrease histologic lesions and normalization of ventilation rate, we infer that the amelioration of the injurious state decreased the need for this protective pathway and so the levels of gene expression declined.

Plasma samples collected as described above were analyzed and MIP-1 alpha and MCP-1, two chemokines associated with acute lung injury, were initially increased with the E coli challenge, but they were significantly reduced by treatment with Compound 1.

### Example 7

### Evaluation of Compound 1 and Compound 2 in the Murine Pneumonia Model

The effects of Compound 1 and Compound 2[2-(3-fluoro-4-hydroxyphenyl)-7-vinyl-1,3-benzoxazol-5-ol, or ERB-041] initially dosed at 3 mg/kg IV were being assessed on 7-day survival, microbial clearance and attenuation the acute and chronic pro-inflammatory effects of invasive pneumococcal pneumonia in lung tissue and distant organs in the murine pneumonia model. [Mohler J et al. Intensive Care Med 2003; 29:808-816.] Compound 1 and Compound 2 were tested to determine their ability to modulate the pathophysiology of severe infection and mortality from local and systemic inflammation from severe bacterial pneumonia. The compounds were delivered IV at 24 and 48 hours after inoculation with S. pneumoniae at an LD₉₀ dose. Moxifloxacin was given at 6, 24 and 48 hours. The animals became bacteremic and develop a lethal pulmonary and systemic infection between 48 and 72 hours after the primary inoculation. All of the vehicle treated animals succumbed by 80 hours (3 days and 8 hours), while at 7 days 20% of the Compound 1—treated animals were alive and 60% of the Compound 2—treated animals survived.

The materials, methods, and examples presented herein are intended to be illustrative, and are not intended to limit the scope of the invention. All publications, including patent applications, patents, Genbank accession records and other references mentioned herein are incorporated by reference in their entirety.

What is claimed is:

1. A method of treating acute lung injury in a subject in need thereof, comprising administering to said subject a therapeutically effective amount of an ERβ selective ligand or a pharmaceutical composition thereof.

2. The method of claim 1 wherein said acute lung injury comprises acute lung injury induced by peritonitis during sepsis, acute lung injury induced by intravenous bacteremia during sepsis, acute lung injury caused by smoke inhalation, acute lung injury occurring in a premature infant with deficiency of surfactant, acute lung injury caused by oxygen toxicity or acute lung injury caused by barotrauma from mechanical ventilation.

3. The method of claim 1 wherein said acute lung injury comprises acute lung injury induced by peritonitis during sepsis, or acute lung injury induced by intravenous bacteremia during sepsis.

4. The method of claim 1 wherein said acute lung injury comprises acute lung injury caused by smoke inhalation.

5. The method of claim 1 wherein said acute lung injury comprises acute lung injury occurring in a premature infant with deficiency of surfactant.

6. The method of claim 1 wherein said acute lung injury comprises acute lung injury caused by oxygen toxicity or acute lung injury caused by barotrauma from mechanical ventilation.

7. The method of claim 1 wherein said acute lung injury comprises acute lung injury caused by oxygen toxicity occurring in a premature infant with deficiency of surfactant, or acute lung injury caused by barotrauma from mechanical ventilation occurring in a premature infant with deficiency of surfactant.

8. A method of treating at least one symptom of acute lung injury in a subject in need thereof, comprising administering to said subject a therapeutically effective amount of an ERβ selective ligand or a pharmaceutical composition thereof.

9. The method of claim 8, wherein the at least one symptom is selected from lung hemorrhage, hyaline membrane formation, pulmonary infiltrates, lung edema, lung inflammation, increased perivascular fluid flux, increased transvascular fluid flux, prevalent interstitial edema, alveolar collapse and increased respiratory rate.

10. The method of claim 8, wherein the at least one symptom is selected from lung edema and lung inflammation.

11. A method of preventing acute lung injury or at least one symptom of acute lung injury in a subject comprising administering to said subject a therapeutically effective amount of an ERβ selective ligand or a pharmaceutical composition thereof wherein said subject is suspected of being at risk for acute lung injury.

12. The method of claim 11 wherein said subject suspected of being at risk for acute lung injury is selected from a subject being suspected of being at risk for sepsis, a subject being suspected of being at risk for severe sepsis, a subject being suspected of being at risk for septic shock, a premature infant with deficiency of surfactant, a subject being suspected of being at risk for inhalation of noxious fumes, a subject being suspected of being at risk for burn, a subject being suspected of being at risk for massive blood transfusion, a subject being suspected of being at risk for acute pancreatitis, and a subject being suspected of being at risk for drug overdose.

13. The method of claim 11, wherein the at least one symptom is selected from lung hemorrhage, hyaline membrane formation, pulmonary infiltrates, lung edema, lung inflammation, increased perivascular fluid flux, increased transvascular fluid flux, prevalent interstitial edema, alveolar collapse and increased respiratory rate.

14. The method of claim 1, wherein the ERβ selective ligand or the pharmaceutical composition thereof is administered orally.

15. The method of claim 1, wherein the ERβ selective ligand or the pharmaceutical composition thereof is administered intravenously.

16. The method of claim 1, wherein the ERβ selective ligand is non-uterotrophic and non-mammotrophic.

17. The method of claim 1, wherein the binding affinity of the ERβ selective ligand to ERβ is at least about 20 times greater than its binding affinity to ERα.

18. The method of claim 1, wherein the subject is a human.

19. The method of claim 1, wherein the ERβ selective ligand has Formula I: 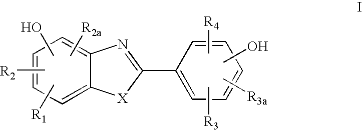
or is a pharmaceutically acceptable salt thereof, wherein:
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms each independently selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, or alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or the ERβ selective ligand has Formula II: 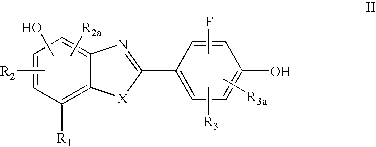
or is a pharmaceutically acceptable salt thereof, wherein:
R₁ is alkenyl of 2-7 carbon atoms; wherein the alkenyl moiety is optionally substituted with hydroxyl, —CN, halogen, trifluoroalkyl, trifluoroalkoxy, —COR, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or the ERβ selective ligand has Formula III: 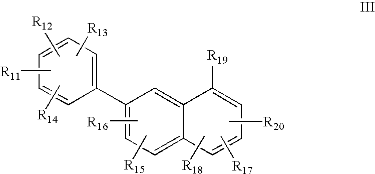
or is a pharmaceutically acceptable salt thereof, wherein:
R₁₁, R₁₂, R₁₃, and R₁₄ are each, independently, selected from hydrogen, hydroxyl, alkyl of 1-6 carbon atoms, alkoxy of 1-6 carbon atoms, or halogen;
R₁₅, R₁₆, R₁₇, R₁₈, R₁₉, and R₂₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms each independently selected from O, N or S; wherein the alkyl or alkenyl moieties of R₁₅, R₁₆, R₁₇, R₁₈, R₁₉, or R₂₀ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈, R₁₉, or R₂₀ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₁, R₁₂, R₁₃, R₁₄, R₁₇, R₁₈, R₁₉ or R₂₀ is hydroxyl, or a pharmaceutically acceptable salt thereof;
or the ERβ selective ligand has Formula IV: 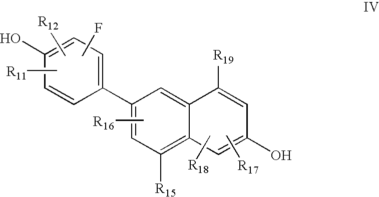
or is a pharmaceutically acceptable salt thereof, wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms each independently selected from O, N or S; wherein the alkyl or alkenyl moieties of R₁₅, R₁₆, R₁₇, R₁₈, or R₁₉ may be optionally substituted with hydroxyl, —CN, halogen, trifluoroalkyl, trifluoroalkoxy, —NO₂, or phenyl; wherein the phenyl moiety of R₁₅, R₁₆, R₁₇, R₁₈, or R₁₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₅ or R₁₉ is not hydrogen;
or the ERβ selective ligand has Formula V: 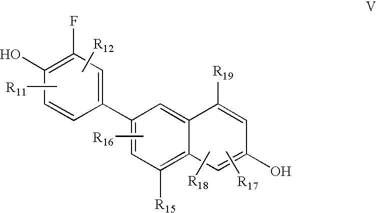
or is a pharmaceutically acceptable salt thereof, wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms each independently selected from O, N or S; wherein the alkyl or alkenyl moieties of R₁₅, R₁₆, R₁₇, R₁₈, or R₁₉ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈ or R₉ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁₅ or R₁₉ is not hydrogen;
or the ERβ selective ligand has Formula VII: 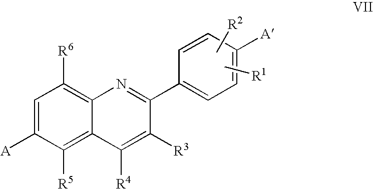
or is a pharmaceutically acceptable salt thereof or a N-oxide thereof, wherein:
A and A′ are each, independently, OH or OP;
P is alkyl, alkenyl, benzyl, acyl, aroyl, alkoxycarbonyl, sulfonyl or phosphoryl;
R¹ and R² are each, independently, H, halogen, C₁-C₆ alkyl, C₂-C₇ alkenyl, or C₁-C₆ alkoxy;
R³ is H, halogen, or C₁-C₆ alkyl;
R⁴ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, or heteroaryl;
R⁵ and R⁶ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R⁴, R⁵ and R⁶ is halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl;
wherein the alkyl or alkenyl moieties of R⁴, R⁵ or R⁶ may be optionally substituted with halogen, OH, —CN, trifluoroalkyl, trifluoroalkoxy, —NO₂, or phenyl;
wherein the alkynyl moiety of R⁴, R⁵ or R⁶ may be optionally substituted with halogen, —CN, —CHO, acyl, trifluoroalkyl, trialkylsilyl, or optionally substituted phenyl;
wherein the phenyl moiety of R⁵ or R⁶ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R⁴, R⁵ and R⁶ are H, C₁-C₆ alkyl, C₂-C₇ alkenyl, or C₁-C₆ alkoxy, then at least one of R¹ and R² is halogen, C₁-C₆ alkyl, C₂-C₇ alkenyl, or C₁-C₆ alkoxy;
provided that at least one of R⁴ and R⁶ is other than H;
or the ERβ selective ligand has Formula X: 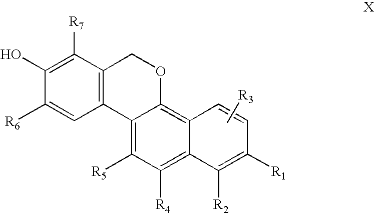
or is a pharmaceutically acceptable salt or prodrug thereof, wherein:
R₁ and R₂ are each, independently, selected from hydrogen, hydroxyl, alkyl of 1-6 carbon atoms, alkenyl of 2-6 carbon atoms, alkynyl of 2-7 carbon atoms, alkoxy of 1-6 carbon atoms, or halogen; wherein the alkyl or alkenyl moieties of R₁, or R₂ may be optionally substituted with hydroxyl, —CN, halogen, trifluoroalkyl, trifluoroalkoxy, —NO₂, or phenyl; and provided that at least one of R₁ or R₂ is hydroxyl;
R₃, R₄, R₅, R₆, and R₇ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms each independently selected from O, N or S; wherein the alkyl or alkenyl moieties of R₄, R₅, R₆, or R₇ may be optionally substituted with hydroxyl, —CN, halogen, trifluoroalkyl, trifluoroalkoxy, —NO₂, or phenyl; wherein the phenyl moiety of R₄ or R₅ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl.

20. The method of claim 1, wherein the ERβ selective ligand has Formula II: 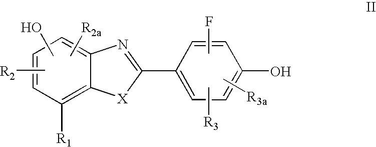
or is a pharmaceutically acceptable salt thereof, wherein:
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently, hydrogen, alkyl of 1-6 carbon atoms, aryl of 6-10 carbon atoms;
X is O, S, or NR₇; and
R₇ is hydrogen, alkyl of 1-6 carbon atoms, aryl of 6-10 carbon atoms, —COR₅, —CO₂R₅ or —SO₂R₅.

21. The method of claim 20, wherein X is O and R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆.

22. The method of claim 20, wherein the ERβ selective ligand has the Formula: 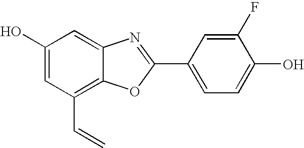
or is a pharmaceutically acceptable salt thereof.

23. The method of claim 1, wherein the ERβ selective ligand has the Formula IV: 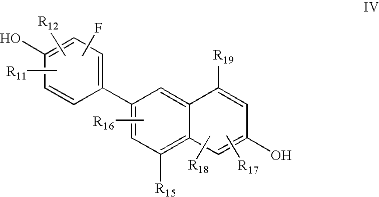
or is a pharmaceutically acceptable salt thereof, wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms each independently selected from O, N or S; wherein the alkyl or alkenyl moieties of R₁₅, R₁₆, R₁₇, R₁₈, or R₁₉ may be optionally substituted with hydroxyl, —CN, halogen, trifluoroalkyl, trifluoroalkoxy, —NO₂, or phenyl; wherein the phenyl moiety of R₁₅, R₁₆, R₁₇, R₁₈, or R₁₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl; and
wherein at least one of R₁₅ or R₁₉ is not hydrogen.

24. The method of claim 1, wherein the ERβ selective ligand has the Formula V: 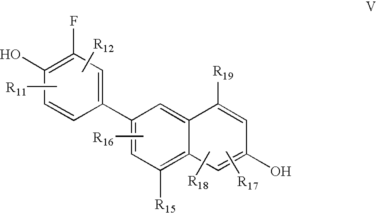
or a pharmaceutically acceptable salt thereof, wherein:
R₁₁ and R₁₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₁₅, R₁₆, R₁₇, R₁₈, and R₁₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms each independently selected from O, N or S; wherein the alkyl or alkenyl moieties of R₁₅, R₁₆, R₁₇, R₁₈, or R₁₉ may be optionally substituted with hydroxyl, CN, halogen, trifluoroalkyl, trifluoroalkoxy, NO₂, or phenyl; wherein the phenyl moiety of R₁₅, R₁₆, R₁₇, R₁₈ or R₉ may be optionally mono-, di-, or tri-substituted with alkyl of 1-6 carbon atoms, alkenyl of 2-7 carbon atoms, halogen, hydroxyl, alkoxy of 1-6 carbon atoms, CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl; and
wherein at least one of R₁₅ or R₁₉ is not hydrogen.

25. The method of claim 24 wherein the 5 or 6-membered heterocyclic ring having 1 to 4 heteroatoms each independently selected from O, N or S is furan, thiophene or pyridine, and R₁₅, R₁₆, R₁₇, R₁₈, and R₁₉ are each, independently, hydrogen, halogen, —CN, or alkynyl of 2-7 carbon atoms.

26. The method of claim 25 wherein R₁₆, R₁₇, and R₁₈ are hydrogen.

27. The method of claim 24, wherein the ERβ selective ligand is a compound having the Formula: 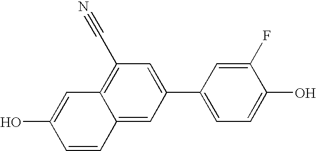
or a pharmaceutically acceptable salt thereof.

28. The method of claim 1, wherein the ERβ selective ligand has Formula X: 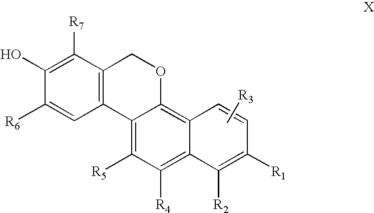
or is a pharmaceutically acceptable salt or prodrug thereof, wherein:
R₁ and R₂ are each, independently, selected from hydrogen, hydroxyl, alkyl of 1-6 carbon atoms, alkenyl of 2-6 carbon atoms, alkynyl of 2-7 carbon atoms, alkoxy of 1-6 carbon atoms, or halogen; wherein the alkyl or alkenyl moieties of R₁, or R₂ may be optionally substituted with hydroxyl, —CN, halogen, trifluoroalkyl, trifluoroalkoxy, —NO₂, or phenyl; and provided that at least one of R₁ or R₂ is hydroxyl;
R₃, R₄, R₅, R₆, and R₇ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms each independently selected from O, N or S; wherein the alkyl or alkenyl moieties of R₄, R₅, R₆, or R₇ may be optionally substituted with hydroxyl, —CN, halogen, trifluoroalkyl, trifluoroalkoxy, —NO₂, or phenyl; wherein the phenyl moiety of R₄ or R₅ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl.

29. The method of claim 28, wherein the ERβ selective ligand is a compound having the formula: 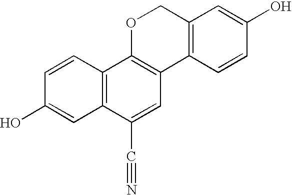
or a pharmaceutically acceptable salt thereof.


SEQUENCE LISTING (3 sequences)

SEQ ID NO: 1 - DNA, 22 bp, Artificial Sequence
  Primer
aatgccttcaacatggaggatt

SEQ ID NO: 2 - DNA, 28 bp, Artificial Sequence
  Primer
ttacaggttccctgtagtttggtattag

SEQ ID NO: 3 - DNA, 21 bp, Artificial Sequence
  Modified Primer
tgtctccgctccagctgccca